(19) United States
(12) Patent Application Publication
(10) Pub. No.: US 2025/0197949 A1
(43) Pub. Date: Jun. 19, 2025

(54) SYSTEMS AND METHODS FOR PREDICTING PROSTATE CANCER RECURRENCE

(71) Applicant: UNIVERSITY OF PITTSBURGH - OF THE COMMONWEALTH SYSTEM OF HIGHER EDUCATION, Pittsburgh, PA (US)

(72) Inventors: Jianhua LUO, Wexford, PA (US); George MICHALOPOULOS, Pittsburgh, PA (US); Joel B. NELSON, Pittsburgh, PA (US); Shuchang LIU, Pittsburgh, PA (US); Yanping YU, Wexford, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 19/031,575

(22) Filed: Jan. 18, 2025

Related U.S. Application Data
(63) Continuation of application No. PCT/US2023/029000, filed on Jul. 28, 2023.
(60) Provisional application No. 63/393,030, filed on Jul. 28, 2022.

(51) Int. Cl. C12Q 1/6886; G16B 40/20; G16H 10/60; G16H 30/40; G16H 50/30

(52) U.S. Cl. CPC C12Q 1/6886; G16B 40/20; G16H 10/60; G16H 30/40; G16H 50/30; C12Q 2600/118; C12Q 2600/156

(57) ABSTRACT

The present disclosure relates to a method of determining whether a subject is at risk of prostate cancer recurrence based on the detection of fusion genes.

### CROSS-REFERENCE TO RELATED APPLICATIONS

This application is a continuation of International Patent Application No. PCT/US2023/029000, filed on Jul. 28, 2023, which claims priority to U.S. Provisional Patent Application Ser. No. 63/393,030, filed on Jul. 28, 2022, which is hereby incorporated by reference in its entirety.

### GRANT INFORMATION

This invention was made with government support under CA229262 and DK120531 awarded by the National Institute of Health, and W81XWH-16-1-0541 awarded by the U.S. Army Medical Research and Materiel Command. The government has certain rights in the invention.

### SEQUENCE LISTING

A Sequence Listing conforming to the rules of WIPO Standard ST.26 is hereby incorporated by reference. Said Sequence Listing has been filed as an electronic document via Patent Center in ASCII format encoded as XML. The electronic document, created on Jan. 18, 2025, is entitled “072396.1054_ST26.xml”, and is 85,972 bytes in size.

### 1. FIELD OF INVENTION

The present invention relates to methods of determining whether a subject is at risk of prostate cancer recurrence based on machine learning models that integrate the fusion gene status of the subject.

### 2. BACKGROUND

Prostate cancer remains one of the most lethal malignancies for men in the United States. Predicting the course of prostate cancer is challenging since only a fraction of prostate cancer patients experienced cancer recurrence after radical prostatectomy or radiation therapy. Currently, Gleason score at the time of diagnosis of prostate cancer is the main criterion in predicting the outcomes of prostate cancer. High Gleason scores, such as combined Gleason scores 8 to 10, are associated with a high risk of prostate cancer recurrence after the radical prostatectomy, while Gleason score 6 is associated with a low risk of recurrence. Indeed, contemporary initial management of Gleason Score 6 is observation (active surveillance and watchful waiting). By combining Gleason score, PSA level, age, and other clinical factors, several prostate cancer nomograms have been developed to gauge the likelihood of prostate cancer recurrence. These tools have achieved variable success in the prediction of prostate cancer clinical outcomes. However, these tools provide little insight into the mechanisms of the disease.

Numerous mutations, gene fusions, chromosome alterations, and epigenetic abnormalities have been discovered in prostate cancer. In prostate cancer, gene-fusion events appear widespread and frequent. Even though some fusion genes such as TMPRSS2-ETS/ERG have been extensively studied, the relationship between gene fusion events and clinical outcomes in patients with prostate cancer remains unclear. Many of these fusion gene products are shed into the bloodstream, and are readily detectable from blood or serum samples of patients. Previous studies have identified 14 fusion genes that are present in prostate cancer samples with various frequencies, ranging from 6% to 80% in the cancer samples. Among these fusion genes, MAN2A1-FER, Pten-NOLC1, and SLC45A2-AMACR are cancer drivers as they induce spontaneous liver cancer in a short period of time when coupled with somatic Pten knockout in the mice. Yet, their potential in predicting the course of prostate cancer is not known. The present disclosure demonstrates the presence of these fusion genes in prostate cancer samples are predictive of prostate cancer behavior. The present disclosure provides methods of determining whether a subject is at risk of prostate cancer recurrence based on machine learning models that integrate the fusion gene status of the subject.

### 3. SUMMARY

The present invention relates to methods for determining whether a subject is at risk of prostate cancer recurrence after radical prostatectomy or radiation therapy. It is based, at least in part, on the results of comprehensive analyses that examined the expression of 14 fusion genes in 607 prostate cancer samples from University of Pittsburgh, Stanford University and University of Wisconsin Madison. The profiling of 14 fusion genes in prostate cancer samples was integrated with Gleason score and serum PSA level to develop machine learning models to predict the recurrence of prostate cancer after radical prostatectomy. The machine learning models were developed by analyzing the data from the University of Pittsburgh cohort as a training set using leave-one-out-cross-validation method. The machine learning models were then applied to the data set from the combined Stanford/Wisconsin cohort as a testing set. The results showed that fusion genes consistently improved the prediction rate of prostate cancer recurrence by Gleason score or serum PSA level or the combination of both. These improvements occurred in both training and testing cohorts and were corroborated by multiple models.

The present invention provides methods for determining whether a subject is at risk of prostate cancer. In certain embodiment, the method comprises: obtaining a sample from a subject, detecting one or more fusion genes in the sample; generating a probability score by a machine learning model, based on an analysis of the one or more fusion genes with respect to fusion genes associated with a reference population; and determining, based on the probability score, the risk of prostate cancer recurrence in the subject.

In certain embodiment, the sample is a blood sample, a serum sample, or a tumor sample. In certain embodiments the sample is processed for RNA isolation.

In certain embodiments, the detection of one or more fusion gene, is determined by reverse transcription polymerase chain reaction (RT-PCR).

In certain embodiments, the one or more fusion genes is selected from the group consisting of MAN2A1-FER, TRMT11-GRIK2, MTOR-TP53BP1, CCNH-05orf30, KDM4B-AC011523.2, SLC45A2-AMACR, TMEM135-CCDC67, LRRC59-F1-160017, CLTC-ETV1, PCMTD1-SNTG1, ACPP-SEC13, DOCK7-OLR1, ZMPSTE24-ZMYM4, Pten-NOLC1, and combinations thereof.

In certain embodiments, the machine learning model comprises one or more machine learning algorithms selected from the group consisting of support vector machine (SVM), random forest (RF), linear discriminant analysis (LDA), logistic regression, or any combination thereof. In certain embodiments, the machine learning model comprises one or more neural networks.

In certain embodiments, the Gleason score or serum PSA level, or combination of both, of the subject are incorporated into the machine learning model.

In certain embodiments, the subject's fusion gene status, Gleason score, PSA level are incorporated into the machine learning model based on leave-one-out cross-validation (LOOCV) analysis of a plurality of training data.

In certain embodiments, the machine learning model is assigned a Gleason score cutoff value of 8. In certain embodiment, the machine learning model is assigned a PSA level cutoff value of 9.77 ng/mL.

In certain embodiments, the subject has received radical prostatectomy or radiation therapy. In certain embodiments, the subject has not received radiation or hormone therapy prior to radical prostatectomy.

In certain embodiments, if the probability score is equal to or less than 0.5 the prostate cancer is predicted as non-recurrent. In certain embodiments, probability score is more than 0.5 the prostate cancer is predicted as recurrent.

In certain embodiments, the machine learning model further accesses prostate cancer imaging data of the subject. Generating the prediction score is further based on an additional analysis of the prostate cancer imaging data by the machine learning model.

In certain embodiments, the machine learning model further accesses biomedical imaging data of the subject. In one feature, the biomedical imaging data comprises one or more of MRI data, X-ray data, ultrasound data, or any combination thereof. Generating the prediction score is further based on an additional analysis of the biomedical imaging data by the machine learning model.

In certain embodiments, the machine learning model further accesses an output from a prostate genome deciper classifier. Generating the prediction score is further based on the output from the prostate genome deciper.

### 4. BRIEF DESCRIPTION OF THE DRAWINGS

For a more complete understanding of the present disclosure and its features and advantages, reference is now made to the following description, taken in conjunction with the accompanying drawings.

FIG. 1 shows that 14 fusion genes, including MAN2A1-FER, TRMT11-GRIK2, MTOR-TP53BP1, CCNH-05orf30, KDM4B-AC011523.2, SLC45A2-AMACR, TMEM135-CCDC67, LRRC59-F1-160017, CLTC-ETV1, PCMTD1-SNTG1, ACPP-SEC13, DOCK7-OLR1, ZMPSTE24-ZMYM4, and Pten-NOLC1, were detected in the prostate cancer samples of the combined cohorts from the University of Pittsburgh Medical Center (UPMC), Stanford University Medical Center and University of Wisconsin Madison Medical center.

FIGS. 2A-2F show the prediction of prostate cancer recurrence by fusion gene profiling, Gleason score, and serum PSA level in the UPMC cohort. FIGS. 2A-2C show the receiver operation characteristic curves from the support vector machine (SVM) model by combining six fusion genes [MAN2A-FER (Ct≤34), TRMT11-GRIK2 (Ct≤43), MTOR-TP53BP1(Ct≤42), CCNH-C5orf30 (negative), PCMTD1-SNTG1 (Ct≤38), and ACPP-SEC13 (Ct≤40), FIG. 2A], Gleason scores (FIG. 2B) or serum PSA levels (FIG. 2C). FIGS. 2D-2F show Kaplan-Meier analyses of PSA-free survival of prostate cancer patients predicted by six fusion gene SVM model (FIG. 2D), Gleason score (FIG. 2E), and serum PSA (FIG. 2F).

FIGS. 3A-3H show fusion genes enhanced predictions by Gleason score, serum PSA level, or the combination of both in the UPMC cohort. FIGS. 3A-3D show receiver operation characteristic curves from six fusion genes [MAN2A-FER (Ct≤34), TRMT11-GRIK2 (Ct≤43), MTOR-TP53BP1(Ct≤42), CCNH-C5orf30 (negative), PCMTD1-SNTG1 (Ct≤38), and ACPP-SEC13 (Ct≤40)]+Gleason SVM model (FIG. 3A), five fusion genes [MAN2A-FER (Ct≤34), MTOR-TP53BP1 (Ct≤42), CCNH-C5orf30 (negative), PCMTD1-SNTG1 (Ct≤38), and ACPP-SEC13 (Ct≤40)]+PSA SVM model (FIG. 3B), Gleason+PSA logistic model (FIG. 3C), three fusion genes [MAN2A-FER (Ct≤34), CCNH-C5orf30 (negative), DOCK7-OLR1 Ct≤41)]+Gleason+PSA random forest model (FIG. 3D). FIGS. 3E-3H show Kaplan-Meier analyses of PSA-free survival of prostate cancer patients predicted by six fusion genes [MAN2A-FER (Ct≤34), TRMT11-GRIK2 (Ct≤43), MTOR-TP53BP1(Ct≤42), CCNH-C5orf30 (negative), PCMTD1-SNTG1 (Ct≤38), and ACPP-SEC13 (Ct≤40)]+Gleason SVM model (FIG. 3E), five fusion genes [MAN2A-FER (Ct≤34), MTOR-TP53BP1 (Ct≤42), CCNH-C5orf30 (negative), PCMTD1-SNTG1 (Ct≤38), and ACPP-SEC13 (Ct≤40)]+PSA SVM model (FIG. 3F), Gleason+PSA logistic model (FIG. 3G), three fusion genes [MAN2A-FER (Ct≤34), CCNH-C5orf30 (negative), DOCK7-OLR1 Ct≤41)]+Gleason+PSA random forest model (FIG. 3H).

FIGS. 4A-4F show fusion gene algorithms from UPMC cohort improved PSA-free survival predictions by Gleason score, serum PSA, or the combination of both in Stanford+Wisconsin cohort. FIGS. 4A-4C show Kaplan-Meier analyses of PSA-free survival of prostate cancer patients in Stanford+Wisconsin cohort predicted by Gleason (cutoff=8, FIG. 4A), PSA (cutoff=9.77 ng/ml, FIG. 4B), or Gleason+PSA (Logistic, FIG. 4C). FIGS. 4D-4F show Kaplan-Meier analyses of PSA-free survival of prostate cancer patients in Stanford+Wisconsin cohort predicted by four fusion genes [TRMT11-GRIK2 (Ct≤43), CCNH-C5orf30 (negative), CLTC-ETV1(Ct≤37), and ACPP-SEC13 (Ct≤40)]+Gleason LDA model (FIG. 4D), three fusion gene [TRMT11-GRIK2 (Ct≤43), CCNH-C5orf30 (negative), and ACPP-SEC13 (Ct≤40)]+PSA logistic model (FIG. 4E), four fusion genes [TRMT11-GRIK2 (Ct≤43), CCNH-C5orf30 (negative), ACPP-SEC13 (Ct≤40) and DOCK7-OLR1 Ct≤41)]+Gleason+PSA LDA model (FIG. 4F).

FIGS. 5A-5F show fusion gene algorithm improves prediction of prostate cancer recurrence in combined cohorts of UPMC, Stanford and Wisconsin by Gleason score, serum PSA level or the combination of both. FIGS. 5A-5C show receiver operation characteristic curves from Gleason (FIG. 5A), PSA (FIG. 5B), or Gleason+PSA Logistic model (FIG. 5C). FIGS. 5D-5F show receiver operation characteristic curves from five fusion genes [MAN2A-FER (Ct≤34), TRMT11-GRIK2 (Ct≤43), MTOR-TP53BP1(Ct≤42), CCNH-C5orf30 (negative), and ACPP-SEC13 (Ct≤40)]+Gleason Random Forest model (FIG. 5D), five fusion genes [MAN2A-FER (Ct≤34), MTOR-TP53BP1(Ct≤42), CCNH-C5orf30 (negative), CLTC-ETV1(Ct≤37), and ACPP-SEC13 (Ct≤40)]+PSA Random Forest model (FIG. 5E), five fusion genes [MAN2A-FER (Ct≤34), TRMT11-GRIK2 (Ct≤43), MTOR-TP53BP1(Ct≤42), CCNH-C5orf30 (negative), and ACPP-SEC13 (Ct≤40)]+Gleason+PSA Random Forest model (FIG. 5F).

FIGS. 6A-6F show fusion gene algorithms enhanced PSA-free survival prediction by Gleason score, serum PSA level, or the combination of both in the combined cohorts of UPMC, Stanford, and Wisconsin. FIGS. 6A-6C show Kaplan-Meier analyses of PSA-free survival of prostate cancer patients in the combined cohorts by Gleason (cutoff=8, FIG. 6A), PSA (cutoff=9.77 ng/ml, FIG. 5B), Gleason+PSA (logistic model, FIG. 6C). FIGS. 6D-6F show Kaplan-Meier analyses of PSA-free survival of prostate cancer patients in the combined cohorts by five fusion genes [MAN2A-FER (Ct≤34), TRMT11-GRIK2 (Ct≤43), MTOR-TP53BP1(Ct≤42), CCNH-C5orf30 (negative), and ACPP-SEC13 (Ct≤40)]+Gleason Random Forest model (FIG. 6D), five fusion genes [MAN2A-FER (Ct≤34), MTOR-TP53BP1(Ct≤42), CCNH-C5orf30 (negative), CLTC-ETV1(Ct≤37), and ACPP-SEC13 (Ct≤40)]+PSA Random Forest model (FIG. 6E), five fusion genes [MAN2A-FER (Ct≤34), TRMT11-GRIK2 (Ct≤43), MTOR-TP53BP1(Ct≤42), CCNH-C5orf30 (negative), and ACPP-SEC13 (Ct≤40)]+Gleason+PSA Random Forest model (FIG. 6F).

FIGS. 7A-7F show fusion genes enhanced predictions by Gleason score, serum PSA level, or the combination of both in the Stanford/Wisconsin cohort. FIG. 7A-7C show receiver operation characteristic curves from Gleason scores (FIG. 7A) or serum PSA levels (FIG. 7B) or a combination of Gleason score and serum PSA level (Logistic model; FIG. 7C). FIGS. 7D-7F show receiver operation characteristic curves from two fusion genes [TRMT11-GRIK2 (Ct≤43) and CCNH-C5orf30 (negative)]+Gleason LDA model (FIG. 7D), three fusion genes [TRMT11-GRIK2 (Ct≤43), CCNH-C5orf30 (negative), and ACPP-SEC13 (Ct≤40)]+PSA logistic model (FIG. 7E), four fusion genes [TRMT11-GRIK2 (Ct≤43), CCNH-C5orf30 (negative), ACPP-SEC13 (Ct≤40) and DOCK7-OLR1 Ct≤41)]+Gleason+PSA LDA model (FIG. 7F).

### 5. DETAILED DESCRIPTION

The present disclosure relates to methods of determining whether a subject is at risk of prostate cancer recurrence following radical prostatectomy or radiation therapy. The present disclosure is based, in part, on the discovery that the detection of a select set of fusion genes provides enhanced prediction of prostate clinical outcomes. Using the methods disclosed herein, the prediction of prostate cancer reoccurrence can be quantified within a degree of certainty.

For clarity, and not by way of limitation, the detailed description of the invention is divided into the following subsections:
5.1 Definitions;
5.2 Fusion genes;
5.3 Fusion gene detection; and
5.4 Methods of determining.

### 5.1 Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

The terms “prostate cancer patient” or “subject having prostate cancer,” as used interchangeably herein, refer to a subject having or who has had a carcinoma of the prostate. The use of the term “patient” does not suggest that the subject has received any treatment for the cancer, but rather that the subject has at some point come to the attention of the healthcare system. The patient/subject, prior to or contemporaneous with the practicing of the invention, may be untreated for prostate cancer, may have received treatment or are currently undergoing treatment, including but not limited to, surgical, chemotherapeutic, anti-androgen or radiologic treatment.

As used herein, the term “disease” refers to any condition or disorder that damages or interferes with the normal function of a cell, tissue, or organ.

As used herein, the term “tumor,” refers to all neoplastic cell growth and proliferation, whether malignant or benign, and all pre-cancerous and cancerous cells and tissues.

The term “recurrence” refers to the detection of prostate cancer in form of metastatic spread of tumor cells, local recurrence, contralateral recurrence or recurrence of prostate cancer at any site of the body of the patient after prostate cancer had been substantially undetectable or responsive to treatments.

The term “nucleic acid molecule” and “nucleotide sequence,” as used herein, refers to a single or double-stranded covalently-linked sequence of nucleotides in which the 3′ and 5′ ends on each nucleotide are joined by phosphodiester bonds. The nucleic acid molecule can include deoxyribonucleotide bases or ribonucleotide bases, and can be manufactured synthetically in vitro or isolated from natural sources.

The term “prognosis” or “predict” refers to a forecast or calculation of risk of developing cancer or a disease or a tumor type, and how a patient will progress, and whether there is a chance of recovery. “Cancer prognosis” generally refers to a forecast or prediction of the probable course or outcome of the cancer and/or patient, assessing the risk of cancer occurrence or recurrence, determining treatment modality, or determining treatment efficacy or responses. Prognosis can use the information of the individual as well as external data to compare against the information of the individual, such as population data, response rate for survivors, family or other genetic information, and the like. “Prognosis” is also used in the context of predicting disease progression, in particular to predict therapeutic results of a certain therapy of the disease, in particular neoplastic conditions, or tumor types. The prognosis of a therapy is used to predict a chance of success (i.e. curing a disease) or chance of reducing the severity of the disease to a certain level. As a general concept, markers screened for this purpose are preferably derived from sample data of patients treated according to the therapy to be predicted. The marker sets may also be used to monitor a patient for the emergence of therapeutic results or positive disease progressions. The term “gene profiling” is used in the broadest sense, and includes methods of quantification of mRNA and/or protein levels in a biological sample.

As used herein, the term “increased risk” refers to an increase in the risk level, for a human subject after testing, for the presence of a cancer relative to a population's known prevalence of a particular cancer before testing.

As used herein, the term “decreased risk” refers to a decrease in the risk level, for a human subject after testing, for the presence of a cancer relative to a population's known prevalence of a particular cancer before testing. In this instance, “decreased risk” refers to a change in risk level relative to a population before testing.

As used herein, the term “cohort” refers to a group or segment of human subjects with shared factors or influences, such as age, family history, cancer risk factors, environmental influences, etc.

As used herein the term, “Receiver Operating Characteristic Curve,” or, “ROC curve,” is a plot of the performance of a particular feature for distinguishing two populations, patients with prostate cancer recurrence, and controls, e.g., those without prostate cancer recurrence. Data across the entire population (namely, the patients and controls) are sorted in ascending order based on the value of a single feature. Then, for each value for that feature, the true positive and false positive rates for the data are determined. The true positive rate is determined by counting the number of cases above the value for that feature under consideration and then dividing by the total number of patients. The false positive rate is determined by counting the number of controls above the value for that feature under consideration and then dividing by the total number of controls.

ROC curves can be generated for a single feature as well as for other single outputs, for example, a combination of two or more features that are combined (such as, added, subtracted, multiplied etc.) to provide a single combined value which can be plotted in a ROC curve.

The ROC curve is a plot of the true positive rate (sensitivity) of a test against the false positive rate (1-specificity) of the test. ROC curves provide another means to quickly screen a data set.

As used herein, the term “specificity” refers to statistical analysis that measures the proportion of negatives which are correctly identified as negative; true negatives. The higher the specificity the lower the false positive rate. The higher the combined specificity and sensitivity the better predictor a fusion gene, or panel of fusion genes, are for correctly identifying prostate cancer recurrence with clinical utility.

As used herein, the term “sensitivity” refers to statistical analysis that measures the proportion of positives which are correctly identified as positives: true positives. The higher the sensitivity the fewer false negatives are identified. The sensitivity, at a designated specificity cutoff, of a fusion gene or panels or fusion genes for a particular disease (e.g., prostate cancer recurrence) can be measured and used to assess a patient's risk for the particular disease.

The term “machine learning model” (or “model”) refers to a collection of parameters and functions, where the parameters are trained on a set of training samples. The parameters and functions may be a collection of linear algebra operations, non-linear algebra operations, and tensor algebra operations. The parameters and functions may include statistical functions, tests, and probability models. The training samples can correspond to samples having measured properties of the sample (e.g., genomic data and other subject data, such as images or health records), as well as known classifications/labels (e.g., phenotypes or treatments) for the subject. The model can learn from the training samples in a training process that optimizes the parameters (and potentially the functions) to provide an optimal quality metric (e.g., accuracy) for classifying new samples. The training function can include expectation maximization, maximum likelihood, Bayesian parameter estimation methods such as markov chain monte carlo, gibbs sampling, hamiltonian monte carlo, and variational inference, or gradient based methods such as stochastic gradient descent and the Broyden-Fletcher-Goldfarb-Shanno (BFGS) algorithm. Example parameters include weights (e.g., vector or matrix transformations) that multiply values, e.g., in regression or neural networks, families of probability distributions, or a loss, cost or objective function that assigns scores and guides model training. Example parameters include weights that multiple values, e.g., in regression or neural networks. A model can include multiple submodels, which may be different layers of a model or independent model, which may have a different structural form, e.g., a combination of a neural network and a support vector machine (SVM). Examples of machine learning models include support vector machines (SVMs), random forest (RF), linear discriminant analysis (LDA), logistic regression and extensions, deep learning models, neural networks (e.g., deep learning neural networks), kernel-based regressions, adaptive basis regression or classification, Bayesian methods, ensemble methods, Gaussian processes, a probabilistic model, and a probabilistic graphical model. A machine learning model can further include feature engineering (e.g., gathering of features into a data structure such as a 1, 2, or greater dimensional vector) and feature representation (e.g., processing of data structure of features into transformed features to use in training for inference of a classification).

The term “features” refers to variables that are used by the model to predict an output classification (label) of a subject, e.g., a condition, or suggested treatments. Values of the variables can be determined for a sample and used to determine a subject classification. Example of input features include a genetic data or medical history.

### 5.2 Fusion Genes

The term “fusion gene,” as used herein, refers to a nucleic acid or protein sequence which combines elements of the recited genes or their RNA transcripts in a manner not found in the wild type/normal nucleic acid or protein sequences. For example, but not by way of limitation, in a fusion gene in the form of genomic DNA, the relative positions of portions of the genomic sequences of the recited genes is altered relative to the wild type/normal sequence (for example, as reflected in the NCBI chromosomal positions or sequences set forth herein). In a fusion gene in the form of mRNA, portions of RNA transcripts arising from both component genes are present (not necessarily in the same register as the wild-type transcript and possibly including portions normally not present in the normal mature transcript). In non-limiting embodiments, such a portion of genomic DNA or mRNA may comprise at least about 10 consecutive nucleotides, or at least about 20 consecutive nucleotides, or at least about 30 consecutive nucleotides, or at least 40 consecutive nucleotides. In certain embodiments, such a portion of genomic DNA or mRNA may comprise up to about 10 consecutive nucleotides, up to about 50 consecutive nucleotides, up to about 100 consecutive nucleotides, up to about 200 consecutive nucleotides, up to about 300 consecutive nucleotides, up to about 400 consecutive nucleotides, up to about 500 consecutive nucleotides, up to about 600 consecutive nucleotides, up to about 700 consecutive nucleotides, up to about 800 consecutive nucleotides, up to about 900 consecutive nucleotides, up to about 1,000 consecutive nucleotides, up to about 1,500 consecutive nucleotides or up to about 2,000 consecutive nucleotides of the nucleotide sequence of a gene present in the fusion gene. In certain embodiments, such a portion of genomic DNA or mRNA may comprise no more than about 10 consecutive nucleotides, about 50 consecutive nucleotides, about 100 consecutive nucleotides, about 200 consecutive nucleotides, about 300 consecutive nucleotides, about 400 consecutive nucleotides, about 500 consecutive nucleotides, about 600 consecutive nucleotides, about 700 consecutive nucleotides, about 800 consecutive nucleotides, about 900 consecutive nucleotides, about 1,000 consecutive nucleotides, about 1,500 consecutive nucleotides or about 2,000 consecutive nucleotides of the nucleotide sequence of a gene present in the fusion gene. In certain embodiments, such a portion of genomic DNA or mRNA does not comprise the full wildtype/normal nucleotide sequence of a gene present in the fusion gene. In a fusion gene in the form of a protein, portions of amino acid sequences arising from both component genes are present (not by way of limitation, at least about 5 consecutive amino acids or at least about 10 amino acids or at least about 20 amino acids or at least about 30 amino acids). In certain embodiments, such a portion of a fusion gene protein may comprise up to about 10 consecutive amino acids, up to about 20 consecutive amino acids, up to about 30 consecutive amino acids, up to about 40 consecutive amino acids, up to about 50 consecutive amino acids, up to about 60 consecutive amino acids, up to about 70 consecutive amino acids, up to about 80 consecutive amino acids, up to about 90 consecutive amino acids, up to about 100 consecutive amino acids, up to about 120 consecutive amino acids, up to about 140 consecutive amino acids, up to about 160 consecutive amino acids, up to about 180 consecutive amino acids, up to about 200 consecutive amino acids, up to about 220 consecutive amino acids, up to about 240 consecutive amino acids, up to about 260 consecutive amino acids, up to about 280 consecutive amino acids or up to about 300 consecutive amino acids of the amino acid sequence encoded by a gene present in the fusion gene. In certain embodiments, such a portion of a fusion gene protein may comprise no more than about 10 consecutive amino acids, about 20 consecutive amino acids, about 30 consecutive amino acids, about 40 consecutive amino acids, about 50 consecutive amino acids, about 60 consecutive amino acids, about 70 consecutive amino acids, about 80 consecutive amino acids, about 90 consecutive amino acids, about 100 consecutive amino acids, about 120 consecutive amino acids, about 140 consecutive amino acids, about 160 consecutive amino acids, about 180 consecutive amino acids, about 200 consecutive amino acids, about 220 consecutive amino acids, about 240 consecutive amino acids, about 260 consecutive amino acids, about 280 consecutive amino acids or about 300 consecutive amino acids of the amino acid sequence encoded by a gene present in the fusion gene. In certain embodiments, such a portion of a fusion gene protein does not comprise the full wildtype/normal amino acid sequence encoded by a gene present in the fusion gene. In this paragraph, portions arising from both genes, transcripts or proteins do not refer to sequences which may happen to be identical in the wild type forms of both genes (that is to say, the portions are “unshared”). As such, a fusion gene represents, generally speaking, the splicing together or fusion of genomic elements not normally joined together. See WO 2015/103057 and WO 2016/011428, the contents of which are hereby incorporated by reference, for additional information regarding the disclosed fusion genes.

The fusion gene TRMT11-GRIK2 is a fusion between the tRNA methyltransferase 11 homolog (“TRMT11”) and glutamate receptor, ionotropic, kainate 2 (“GRIK2”) genes. The human TRMT11 gene is typically located on chromosome 6q11.1 and the human GRIK2 gene is typically located on chromosome 6q16.3. In certain embodiments, the TRMT11 gene is the human gene having NCBI Gene ID No: 60487, sequence chromosome 6; NC_000006.11 (126307576 . . . 126360422) and/or the GRIK2 gene is the human gene having NCBI Gene ID No:2898, sequence chromosome 6; NC_000006.11 (101841584 . . . 102517958).

The fusion gene SLC45A2-AMACR is a fusion between the solute carrier family 45, member 2 (“SLC45A2”) and alpha-methylacyl-CoA racemase (“AMACR”) genes. The human SLC45A2 gene is typically located on human chromosome 5p13.2 and the human AMACR gene is typically located on chromosome 5p13. In certain embodiments the SLC45A2 gene is the human gene having NCBI Gene ID No: 51151, sequence chromosome 5; NC_000005.9 (33944721 . . . 33984780, complement) and/or the AMACR gene is the human gene having NCBI Gene ID No:23600, sequence chromosome 5; NC_000005.9 (33987091 . . . 34008220, complement).

The fusion gene MTOR-TP53BP1 is a fusion between the mechanistic target of rapamycin (“MTOR”) and tumor protein p53 binding protein 1 (“TP53BP1”) genes. The human MTOR gene is typically located on chromosome 1p36.2 and the human TP53BP1 gene is typically located on chromosome 15q15-q21. In certain embodiments, the MTOR gene is the human gene having NCBI Gene ID No:2475, sequence chromosome 1 NC_000001.10 (11166588 . . . 11322614, complement) and/or the TP53BP1gene is the human gene having NCBI Gene ID No: 7158, sequence chromosome 15; NC_000015.9 (43695262 . . . 43802707, complement).

The fusion gene LRRC59-FLJ60017 is a fusion between the leucine rich repeat containing 59 (“LRRC59”) gene and the “FLJ60017” nucleic acid. The human LRRC59 gene is typically located on chromosome 17q21.33 and nucleic acid encoding human FLJ60017 is typically located on chromosome 11q12.3. In certain embodiments, the LRRC59 gene is the human gene having NCBI Gene ID No:55379, sequence chromosome 17; NC_000017.10 (48458594 . . . 48474914, complement) and/or FLJ60017 has a nucleic acid sequence as set forth in GeneBank AK_296299.

The fusion gene TMEM135-CCDC67 is a fusion between the transmembrane protein 135 (“TMEM135”) and coiled-coil domain containing 67 (“CCDC67”) genes. The human TMEM135 gene is typically located on chromosome 11q14.2 and the human CCDC67 gene is typically located on chromosome 11q21. In certain embodiments the TMEM135 gene is the human gene having NCBI Gene ID No: 65084, sequence chromosome 11; NC_000011.9 (86748886 . . . 87039876) and/or the CCDC67 gene is the human gene having NCBI Gene ID No: 159989, sequence chromosome 11; NC_000011.9 (93063156 . . . 93171636).

The fusion gene CCNH-C5orf30 is a fusion between the cyclin H (“CCNH”) and chromosome 5 open reading frame 30 (“C5orf30”) genes. The human CCNH gene is typically located on chromosome 5q13.3-q14 and the human C5orf30gene is typically located on chromosome 5q21.1. In certain embodiments, the CCNH gene is the human gene having NCBI Gene ID No: 902, sequence chromosome 5; NC_000005.9 (86687310 . . . 86708850, complement) and/or the C5orf30gene is the human gene having NCBI Gene ID No: 90355, sequence chromosome 5; NC_000005.9 (102594442 . . . 102614361).

The fusion gene KDM4B-AC011523.2 is a fusion between lysine (K)-specific demethylase 4B (“KDM4B”) and chromosomal region “AC011523.2.” The human KDM4B gene is typically located on chromosome 19p13.3 and the human AC011523.2 region is typically located on chromosome 19q13.4. In certain embodiments the KDM4B gene is the human gene having NCBI Gene ID NO: 23030, sequence chromosome 19; NC_000019.9 (4969123 . . . 5153609).

The fusion gene MAN2A1-FER is a fusion between mannosidase, alpha, class 2A, member 1 (“MAN2A1”) and (fps/fes related) tyrosine kinase (“FER”). The human MAN2A1 gene is typically located on chromosome 5q21.3 and the human FER gene is typically located on chromosome 5q21. In certain embodiments, the MAN2A1gene is the human gene having NCBI Gene ID NO: 4124, sequence chromosome 5; NC_000005.9 (109025156 . . . 109203429) or NC_000005.9 (109034137 . . . 109035578); and/or the FER gene is the human gene having NCBI Gene ID NO: 2241, sequence chromosome 5: NC_000005.9 (108083523 . . . 108523373).

The fusion gene PTEN-NOLC1 is a fusion between the phosphatase and tensin homolog (“PTEN”) and nucleolar and coiled-body phosphoprotein 1 (“NOLC1”). The human PTEN gene is typically located on chromosome 10q23.3 and the human NOLC1 gene is typically located on chromosome 10q24.32. In certain embodiments, the PTEN gene is the human gene having NCBI Gene ID NO: 5728, sequence chromosome 10; NC_000010.11 (87863438 . . . 87970345) and/or the NOLC1 gene is the human gene having NCBI Gene ID NO: 9221, sequence chromosome 10; NC_000010.11 (102152176 . . . 102163871).

The fusion gene ZMPSTE24-ZMYM4 is a fusion between zinc metallopeptidase STE24 (“ZMPSTE24”) and zinc finger, MYM-type 4 (“ZMYM4”). The human ZMPSTE24 is typically located on chromosome 1p34 and the human ZMYM4 gene is typically located on chromosome 1p32-p34. In certain embodiments, the ZMPSTE24 gene is the human gene having NCBI Gene ID NO: 10269, sequence chromosome 1; NC_000001.11 (40258050 . . . 40294184) and/or the ZMYM4 gene is the human gene having NCBI Gene ID NO: 9202, sequence chromosome 1; NC_000001.11 (35268850 . . . 35421944).

The fusion gene CLTC-ETV1 is a fusion between clathrin, heavy chain (Hc) (“CLTC”) and ets variant 1 (“ETV1”). The human CLTC is typically located on chromosome 17q23.1 and the human ETV1 gene is typically located on chromosome 7p21.3. In certain embodiments, the CLTC gene is the human gene having NCBI Gene ID NO: 1213, sequence chromosome 17; NC_000017.11 (59619689 . . . 59696956) and/or the ETV1gene is the human gene having NCBI Gene ID NO: 2115, sequence chromosome 7; NC_000007.14 (13891229 . . . 13991425, complement).

The fusion gene ACPP-SEC13 is a fusion between acid phosphatase, prostate (“ACPP”) and SEC13 homolog (“SEC13”). The human ACPP is typically located on chromosome 3q22.1 and the human SEC13 gene is typically located on chromosome 3p25-p24. In certain embodiments, the ACPP gene is the human gene having NCBI Gene ID NO: 55, sequence chromosome 3; NC_000003.12 (132317367 . . . 132368302) and/or the SEC13 gene is the human gene having NCBI Gene ID NO: 6396, sequence chromosome 3; NC_000003.12 (10300929 . . . 10321188, complement).

The fusion gene DOCK7-OLR1 is a fusion between dedicator of cytokinesis 7 (“DOCK7”) and oxidized low density lipoprotein (lectin-like) receptor 1 (“OLR1”). The human DOCK7 is typically located on chromosome 1p31.3 and the human OLR1 gene is typically located on chromosome 12p13.2-p12.3. In certain embodiments, the DOCK7 gene is the human gene having NCBI Gene ID NO: 85440, sequence chromosome 1; NC_000001.11 (62454726 . . . 62688368, complement) and/or the OLR1 gene is the human gene having NCBI Gene ID NO: 4973, sequence chromosome 12; NC_000012.12 (10158300 . . . 10172191, complement).

The fusion gene PCMTD1-SNTG1 is a fusion between protein-L-isoaspartate (D-aspartate)O-methyltransferase domain containing 1 (“PCMTD1”) and syntrophin, gamma 1 (“SNTG1”). The human PCMTD1 is typically located on chromosome 8q11.23 and the human SNTG1 gene is typically located on chromosome 811.21. In certain embodiments, the PCMTD1 gene is the human gene having NCBI Gene ID NO: 115294, sequence chromosome 8; NC_000008.11 (51817575 . . . 51899186, complement) and/or the SNTG1gene is the human gene having NCBI Gene ID NO: 54212, sequence chromosome 8; NC_000008.11 (49909789 . . . 50794118).

### 5.3 Fusion Gene Detection

Any of the foregoing fusion genes described above in section 5.2 may be identified and/or detected by methods known in the art. The fusion genes may be detected by detecting a fusion gene manifested in a DNA molecule, an RNA molecule or a protein. In certain embodiments, a fusion gene can be detected by determining the presence of a DNA molecule, an RNA molecule or protein that is encoded by the fusion gene. For example, and not by way of limitation, the presence of a fusion gene may be detected by determining the presence of the protein encoded by the fusion gene.

The fusion gene may be detected in a sample of a subject. A “patient” or “subject,” as used interchangeably herein, refers to a human or a non-human subject. Non-limiting examples of non-human subjects include non-human primates, dogs, cats, mice, etc. The subject may or may not be previously diagnosed as having cancer.

In certain non-limiting embodiments, a sample includes, but is not limited to, cells in culture, cell supernatants, cell lysates, serum, blood plasma, biological fluid (e.g., blood, plasma, serum, stool, urine, lymphatic fluid, ascites, ductal lavage, saliva and cerebrospinal fluid) and tissue samples. The source of the sample may be solid tissue (e.g., from a fresh, frozen, and/or preserved organ, tissue sample, biopsy, or aspirate), blood or any blood constituents, bodily fluids (such as, e.g., urine, lymph, cerebral spinal fluid, amniotic fluid, peritoneal fluid or interstitial fluid), or cells from the individual, including circulating cancer cells. In certain non-limiting embodiments, the sample is obtained from a cancer. In certain embodiments, the sample may be a “biopsy sample” or “clinical sample,” which are samples derived from a subject. In certain embodiments, the sample includes one or more cancer cells from a subject. In certain embodiments, the one or more fusion genes can be detected in one or more samples obtained from a subject, e.g., in one or more cancer cell samples. In certain embodiments, the sample is a blood sample, e.g., buffy coat sample, from a subject. In certain embodiments, the sample is not a prostate cancer sample or one or more prostate cancer cells.

In certain non-limiting embodiments, the fusion gene is detected by nucleic acid hybridization analysis.

In certain non-limiting embodiments, the fusion gene is detected by fluorescent in situ hybridization (FISH) analysis. FISH is a technique that can directly identify a specific sequence of DNA or RNA in a cell or biological sample and enables visual determination of the presence and/or expression of a fusion gene in a tissue sample. In certain non-limiting embodiments, where a fusion gene combines genes not typically present on the same chromosome, FISH analysis may demonstrate probes binding to the same chromosome. For example, and not by way of limitation, analysis may focus on the chromosome where one gene normally resides and then hybridization analysis may be performed to determine whether the other gene is present on that chromosome as well.

In certain non-limiting embodiments, the fusion gene is detected by DNA hybridization, such as, but not limited to, Southern blot analysis.

In certain non-limiting embodiments, the fusion gene is detected by RNA hybridization, such as, but not limited to, Northern blot analysis. In certain embodiments, Northern blot analysis can be used for the detection of a fusion gene, where an isolated RNA sample is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography to detect the presence of a fusion gene in the RNA sample.

In certain non-limiting embodiments, the fusion gene is detected by nucleic acid sequencing analysis.

In certain non-limiting embodiments, the fusion gene is detected by probes present on a DNA array, chip or a microarray. For example, and not by way of limitation, oligonucleotides corresponding to one or more fusion genes can be immobilized on a chip which is then hybridized with labeled nucleic acids of a sample obtained from a subject. Positive hybridization signal is obtained with the sample containing the fusion gene transcripts.

In certain non-limiting embodiments, the fusion gene is detected by a method comprising Reverse Transcription Polymerase Chain Reaction (“RT-PCR”). In certain embodiments, the fusion gene is detected by a method comprising RT-PCR using the one or more pairs of primers disclosed herein (see, for example, Table 9).

In certain non-limiting embodiments, the fusion gene is detected by antibody binding analysis such as, but not limited to, Western Blot analysis and immunohistochemistry.

### 5.4 Methods of Determining

The present invention provides methods of determining whether a subject is at risk of prostate cancer recurrence, the methods including: obtaining a sample from the subject, determining the fusion gene status of the subject, integrating the subject fusion gene status into a machine learning model, determining the risk of prostate cancer recurrence in the subject.

In certain non-limiting embodiments, the method of determining whether a subject is at risk of prostate cancer recurrence comprises obtaining a sample from the subject. In certain embodiments the sample is a tumor sample or blood sample.

In certain non-limiting embodiments, the method of determining whether a subject is at risk of prostate cancer recurrence comprises determining the fusion gene status of the subject. In certain embodiment, the fusion gene status of the subject, comprises determining whether a sample of the subject contains one or more fusion genes selected from the group consisting of MAN2A1-FER, TRMT11-GRIK2, MTOR-TP53BP1, CCNH-05orf30, KDM4B-AC011523.2, SLC45A2-AMACR, TMEM135-CCDC67, LRRC59-F1-160017, CLTC-ETV1, PCMTD1-SNTG1, ACPP-SEC13, DOCK7-OLR1, ZMPSTE24-ZMYM4, and Pten-NOLC1 or a combination thereof, where the presence of one or more fusion genes in the sample is indicative that the fusion gene status of the subject. In certain embodiments, the fusion gene status of a subject is indicative of prostate cancer recurrence.

In certain embodiments, the method of determining whether a subject is at risk of prostate cancer recurrence comprises determining the presence and/or absence of one or more, two or more, three or more, four or more, five or more, six or more, seven or more, eight or more, nine or more, ten or more, eleven or more, twelve or more, thirteen or more, fourteen or more of the fusion genes disclosed herein in a sample of a subject.

In certain embodiments, the method of determining whether a subject is at risk prostate cancer recurrence comprises determining whether a sample of the subject contains one or more fusion genes selected from the group consisting of MAN2A1-FER, TRMT11-GRIK2, MTOR-TP53BP1, PCMTD1-SNTG1, ACPP-SEC13, DOCK7-OLR1, and combinations thereof, where the presence of one or more fusion genes in the sample is indicative that the subject is at risk of prostate cancer recurrence.

In certain embodiments, the method of determining whether a subject is at risk of prostate cancer recurrence comprises a machine learning model, where the machine learning model integrates the fusion gene status of a subject and generates a prediction probability. In certain embodiments, the method of determining whether the subject is at risk of prostate cancer recurrence further comprises transforming the one or more detected fusion genes into one or more embeddings and inputting the one or more embeddings into the machine learning model. In certain embodiments, the machine learning model comprises one or more machine learning algorithms, wherein the one or more machine learning algorithms are selected from the group consisting of support vector machine (SVM), random forest (RF), linear discriminant analysis (LDA), logistic regression or combination thereof. In certain embodiments, the machine learning model is a deep learning model. As an example and not by way of limitation, the deep learning model can comprise convolutional neural networks.

In certain embodiments the machine learning model can be combined with other known prediction methods, including Prostate Imaging Reporting and Data System (PI-RADS) and prostate genome decipher classifier, to further improve the prediction further. In certain embodiments, the machine learning model can be combined with biomedical imaging data, including, but not limited to, data obtained through MRI, X-ray, ultrasound, or other biomedical imaging methods.

In certain embodiments, the machine learning model further integrates a subject's clinical features such as Gleason score and serum PSA. In certain embodiments, the machine learning model further integrates data obtained from the subject's prostate cancer imaging records. In certain embodiments, the subjects fusion gene status, Gleason score, PSA level are incorporated into the machine learning model by leave-one-out cross-validation (LOOCV) analysis.

In certain embodiments, a probability of more than 0.5, is deemed recurrent. In certain embodiments, a probability equal to or less than 0.5 is non-recurrent.

In certain embodiments, the machine learning model is assigned a Gleason score cutoff value of 8. In certain embodiment, the machine learning model is assigned a PSA level cutoff value of 9.77 ng/mL.

In certain embodiments, the subject has received radical prostatectomy or radiation therapy. In certain embodiments, the subject has not received radiation or hormone therapy prior to radical prostatectomy.

### 5.5 Computer System

In certain embodiments, the machine learning model is implemented in a computer system. Figure ˜illustrates an example computer system ˜00. In particular embodiments, one or more computer systems ˜00 perform one or more steps of one or more methods described or illustrated herein. In particular embodiments, one or more computer systems ˜00 provide functionality described or illustrated herein. In particular embodiments, software running on one or more computer systems ˜00 performs one or more steps of one or more methods described or illustrated herein or provides functionality described or illustrated herein. Particular embodiments include one or more portions of one or more computer systems ˜00. Herein, reference to a computer system may encompass a computing device, and vice versa, where appropriate. Moreover, reference to a computer system may encompass one or more computer systems, where appropriate.

This disclosure contemplates any suitable number of computer systems ˜00. This disclosure contemplates computer system ˜00 taking any suitable physical form. As example and not by way of limitation, computer system ˜00 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system ˜00 may include one or more computer systems ˜00; be unitary or distributed; span multiple locations; span multiple machines; span multiple data centers; or reside in a cloud, which may include one or more cloud components in one or more networks. Where appropriate, one or more computer systems ˜00 may perform without substantial spatial or temporal limitation one or more steps of one or more methods described or illustrated herein. As an example and not by way of limitation, one or more computer systems ˜00 may perform in real time or in batch mode one or more steps of one or more methods described or illustrated herein. One or more computer systems ˜00 may perform at different times or at different locations one or more steps of one or more methods described or illustrated herein, where appropriate.

In particular embodiments, computer system ˜00 includes a processor ˜02, memory ˜04, storage ˜06, an input/output (I/O) interface ˜08, a communication interface ˜10, and a bus ˜12. Although this disclosure describes and illustrates a particular computer system having a particular number of particular components in a particular arrangement, this disclosure contemplates any suitable computer system having any suitable number of any suitable components in any suitable arrangement.

In particular embodiments, processor ˜02 includes hardware for executing instructions, such as those making up a computer program. As an example and not by way of limitation, to execute instructions, processor ˜02 may retrieve (or fetch) the instructions from an internal register, an internal cache, memory ˜04, or storage ˜06; decode and execute them; and then write one or more results to an internal register, an internal cache, memory ˜04, or storage ˜06. In particular embodiments, processor ˜02 may include one or more internal caches for data, instructions, or addresses. This disclosure contemplates processor ˜02 including any suitable number of any suitable internal caches, where appropriate. As an example and not by way of limitation, processor ˜02 may include one or more instruction caches, one or more data caches, and one or more translation lookaside buffers (TLBs). Instructions in the instruction caches may be copies of instructions in memory ˜04 or storage ˜06, and the instruction caches may speed up retrieval of those instructions by processor ˜02. Data in the data caches may be copies of data in memory ˜04 or storage ˜06 for instructions executing at processor ˜02 to operate on; the results of previous instructions executed at processor ˜02 for access by subsequent instructions executing at processor ˜02 or for writing to memory ˜04 or storage ˜06; or other suitable data. The data caches may speed up read or write operations by processor ˜02. The TLBs may speed up virtual-address translation for processor ˜02. In particular embodiments, processor ˜02 may include one or more internal registers for data, instructions, or addresses. This disclosure contemplates processor ˜02 including any suitable number of any suitable internal registers, where appropriate. Where appropriate, processor ˜02 may include one or more arithmetic logic units (ALUs); be a multi-core processor; or include one or more processors ˜02. Although this disclosure describes and illustrates a particular processor, this disclosure contemplates any suitable processor.

In particular embodiments, memory ˜04 includes main memory for storing instructions for processor ˜02 to execute or data for processor ˜02 to operate on. As an example and not by way of limitation, computer system ˜00 may load instructions from storage ˜06 or another source (such as, for example, another computer system ˜00) to memory ˜04. Processor ˜02 may then load the instructions from memory ˜04 to an internal register or internal cache. To execute the instructions, processor ˜02 may retrieve the instructions from the internal register or internal cache and decode them. During or after execution of the instructions, processor ˜02 may write one or more results (which may be intermediate or final results) to the internal register or internal cache. Processor ˜02 may then write one or more of those results to memory ˜04. In particular embodiments, processor ˜02 executes only instructions in one or more internal registers or internal caches or in memory ˜04 (as opposed to storage ˜06 or elsewhere) and operates only on data in one or more internal registers or internal caches or in memory ˜04 (as opposed to storage ˜06 or elsewhere). One or more memory buses (which may each include an address bus and a data bus) may couple processor ˜02 to memory ˜04. Bus ˜12 may include one or more memory buses, as described below. In particular embodiments, one or more memory management units (MMUs) reside between processor ˜02 and memory ˜04 and facilitate accesses to memory ˜04 requested by processor ˜02. In particular embodiments, memory ˜04 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory ˜04 may include one or more memories ˜04, where appropriate. Although this disclosure describes and illustrates particular memory, this disclosure contemplates any suitable memory.

In particular embodiments, storage ˜06 includes mass storage for data or instructions. As an example and not by way of limitation, storage ˜06 may include a hard disk drive (HDD), a floppy disk drive, flash memory, an optical disc, a magneto-optical disc, magnetic tape, or a Universal Serial Bus (USB) drive or a combination of two or more of these. Storage ˜06 may include removable or non-removable (or fixed) media, where appropriate. Storage ˜06 may be internal or external to computer system ˜00, where appropriate. In particular embodiments, storage ˜06 is non-volatile, solid-state memory. In particular embodiments, storage ˜06 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage ˜06 taking any suitable physical form. Storage ˜06 may include one or more storage control units facilitating communication between processor ˜02 and storage ˜06, where appropriate. Where appropriate, storage ˜06 may include one or more storages ˜06. Although this disclosure describes and illustrates particular storage, this disclosure contemplates any suitable storage.

In particular embodiments, I/O interface ˜08 includes hardware, software, or both, providing one or more interfaces for communication between computer system ˜00 and one or more I/O devices. Computer system ˜00 may include one or more of these I/O devices, where appropriate. One or more of these I/O devices may enable communication between a person and computer system ˜00. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces ˜08 for them. Where appropriate, I/O interface ˜08 may include one or more device or software drivers enabling processor ˜02 to drive one or more of these I/O devices. I/O interface ˜08 may include one or more I/O interfaces ˜08, where appropriate. Although this disclosure describes and illustrates a particular I/O interface, this disclosure contemplates any suitable I/O interface.

In particular embodiments, communication interface ˜10 includes hardware, software, or both providing one or more interfaces for communication (such as, for example, packet-based communication) between computer system ˜00 and one or more other computer systems ˜00 or one or more networks. As an example and not by way of limitation, communication interface ˜10 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface ˜10 for it. As an example and not by way of limitation, computer system ˜00 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system ˜00 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system ˜00 may include any suitable communication interface ˜10 for any of these networks, where appropriate. Communication interface ˜10 may include one or more communication interfaces ˜10, where appropriate. Although this disclosure describes and illustrates a particular communication interface, this disclosure contemplates any suitable communication interface.

In particular embodiments, bus ˜12 includes hardware, software, or both coupling components of computer system ˜00 to each other. As an example and not by way of limitation, bus ˜12 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus ˜12 may include one or more buses ˜12, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

### 6. EXAMPLES

The presently disclosed subject matter will be better understood by reference to the following Examples, which are provided as exemplary of the presently disclosed subject matter, and not by way of limitation.

### Example 1

The present Example is directed to methods of determining whether a subject is at risk of prostate cancer recurrence. The present invention demonstrates that the incorporation of fusion gene status into the prostate cancer diagnostic scheme benefits the patients in diagnosis, prognosis, cancer progression surveillance, and treatment.

The present Example evaluated the expression of 14 fusion genes in 607 prostate cancer samples obtained from the University of Pittsburgh, Stanford University and University of Wisconsin Madison. The expression profile of 14 fusion genes in prostate cancer samples was integrated with Gleason score and serum PSA level to develop machine learning models to predict the recurrence of prostate cancer after radical prostatectomy. The machine learning models were developed by analyzing the data from the University of Pittsburgh cohort as a training set using leave-one-out-cross-validation method. These models were then applied to the data set from the combined Stanford/Wisconsin cohort as a testing set

### Methods

Tissue samples. There were total of 607 prostate cancer tissue specimens in the study from University of Pittsburgh Medical Center (UPMC), Stanford University Medical Center and University of Wisconsin Madison Medical Center. The sample size was estimated by power analysis and (293 on 80% versus 70% comparison) availability of the clinical specimens. Samples from patients who received radiation or hormone therapy prior to radical prostatectomy were excluded. The samples from UPMC were obtained from the University of Pittsburgh Tissue Bank in compliance with institutional regulatory guidelines and comprised 301 PCa samples, including 271 PCa samples with annotated clinical information. The recurrence status of prostate cancer was defined as a serum PSA level of >0.2 ng/mL on at least two consecutive tests obtained after radical prostatectomy. All the human samples in the experiments were obtained in accordance with the guidelines approved by the institutional review board of University of Pittsburgh. All methods were carried out in accordance with relevant guidelines and regulations. Informed-consent exemptions were obtained from University of Pittsburgh Institutional Review Board. All cancer samples were macrodissected. Samples with at least 50% cancer cells were included in the study. The age of patients ranged from 46-73. Since prostate cancer is male specific, no female patients were admitted to the study. Caucasian patients comprised 97% of the study subjects, while African American or black 3%. The surgical procedures were performed between 2000 and 2016. All prostate cancer patients had no prior hormonal or chemotherapy. All cancer samples were macrodissected. Prostate cancer tissues obtained from other institutes included 112 PCa samples from Stanford University, and 194 samples from the University of Wisconsin Madison. The procedure of obtaining the sample tissues was in full compliance with the guidelines of those institutions.

RNA extraction, cDNA synthesis, and TaqMan RT-PCR. The procedures for RNA extraction, cDNA synthesis, and fusion gene detection were similar to those described previously (1, 4, 5, 6, 7, 9, and 10-14). Briefly, total RNA from the cells was extracted using Trizol (Invitrogen, Inc, CA). The quality of the extracted RNA was assessed through 260/280 and 260/230 ratio analyses by Nanodrop™ spectrophotometer (Thermo Fisher Scientific, MA). The samples passing the quality control were accepted for further analysis. The first stranded cDNA was synthesized from ˜2 μg of the total RNA template from each sample. Random hexamers and Superscript II™ (Invitrogen, Inc, CA) were incubated with the RNA at 42° C. for 2 hours. One microliter of each cDNA sample was used for the TaqMan PCR reactions with 50 heat cycles as follows: 94° C. for 30 seconds, 61° C. for 30 seconds, and 72° C. for 30 seconds, using the primers and probes listed in Table 1. The PCR reactions were performed in a thermocycler (QuantStudio 3 real-time PCR system, Thermofisher, Inc or Mastercycler® RealPlex2, Eppendorf, Inc). The 50 cycle is a standard clinical procedure for detecting fusion transcripts in highly fragmented RNA and suboptimal tissue samples. A negative control with no DNA template and a synthetic positive control were included in each batch of reactions. Samples with a cycle threshold (Ct) of 45 were considered positive for fusion gene detection, while those with a Ct of >45 were considered negative. Negative control and a synthetic positive control were included in each batch of the reactions. The PCR products from 8 to 100% of the positive samples were sequenced to verify the fusion genes using the Sanger sequencing method.

TABLE 1
Primers and probes

| Fusion gene | Primers | Probe |
|---|---|---|
| MAN2A1- | F: 5′- | 5′-/56- |
| FER | AGCGCAGTTTGGGATACA | FAM/TCAGAAACA/ZEN/GCCTATGAGG |
| | GCA-3′ | GAAATT/3IABKFQ/-3′ |
| | R: 5′- | |
| | CTTTAATGTGCCCTTATAT | |
| | ACTTCACC-3′ | |
| SLC45A2- | F: 5′- | 5′-/56- |
| AMACR | TTGATGTCTGCTCCCATC | FAM/AAGAGGGCA/ZEN/TGGTAGTGGA |
| | AGG-3′ | GGC/3IABKFQ/-3′ |
| | R: 5′- | |
| | CAGCTGGAGTTTCTCCAT | |
| | GAC-3′ | |
| CCNH- | F: 5′- | 5′-/56- |
| C5orf30 | AAAGTTATTTATCAGAGA | FAM/ACAGGCAAG/ZEN/TTCTGTTCTCT |
| | GTCTGATGCTG-3′ | TTCAGCA/3IABKFQ/-3′ |
| | R: 5′- | |
| | CTGTTCTACTCCAGGTAT | |
| | TTTCATTATATC-3′ | |
| MTOR- | F: 5′- | 5′-/56- |
| TP53BP1 | TGATAGACCAGTCCCGGG | FAM/TGTCAGCCT/ZEN/GTCAGAATCCA |
| | ATG-3′ | AGTCAAG/3IABKFQ/-3′ |
| | R: 5′- | |
| | CCACTGACATTCCCAGAA | |
| | CAAG-3′ | |
| TRMT11- | F: 5′- | 5′-/56- |
| GRIK2 | CCCTTAACAGGTATCTGC | FAM/CGGAACTCC/ZEN/AGATGCTCCTG |
| | TCC-3′ | CG/3IABKFQ/-3′ |
| | R: 5′- | |
| | CCCATTGGGCCAGATTCC | |
| | ACA-3′ | |
| LRRC59- | F: 5′- | 5′-/56- |
| FLJ60017 | GTGACTGCTTGGATGAGA | FAM/CAGTGTGCA/ZEN/AACAAGGTGA |
| | AGC-3′ | CTGGAAG/3IABkFQ/-3′ |
| | R: 5′- | |
| | GTTGATGAGCAGCCATTG | |
| | AGC-3′ | |
| TMEM135- | F: 5′- | 5′-/56- |
| CCDC67 | GAGACCATCTTACTGGAA | FAM/TTTGCCCTT/ZEN/GGTGAGTCTTA |
| | GTTCC-3′ | AAAGGAAC/3IABKF/-3′ |
| | R: 5′- | |
| | TGGTACTCTTCCACCTGTT | |
| | GG-3′ | |
| KDM4B- | F: 5′- | 5′-/56- |
| AC011523.2 | CACACCGAGGACATGGA | FAM/ACAGCATCA/ZEN/ACTACCTGCAC |
| | CCT-3′ | TTTGGG/3IABKFQ/-3′ |
| | R: 5′- | |
| | CTCAGATCCAGGCTTGCT | |
| | TAC-3′ | |
| CLTC- | F: 5′- | 5′-/56- |
| ETV1 | CCTTCCTCCTACATGGAA | FAM/CTGCCAATA/ZEN/CTAGTGTGGCT |
| | GTTG-3′ | TTT/3IABKFQ/-3′ |
| | R: 5′- | |
| | CTTGATTTTCAGTGGCAG | |
| | GCC-3′ | |
| PCMTD1- | F: 5′- | 5′-/56- |
| SNTG1 | CTGGAGAGCTTCATCAAA | FAM/AGCTTTGAT/ZEN/AAACTGCTCTC |
| | AATAG-3′ | CAGAATGTTG/3IABKFQ/-3′ |
| | R: 5′- | |
| | CACTTCTCGGGCAATCTC | |
| | AACA-3′ | |
| ACPP | F: 5′- | 5′-/56- |
| SEC13 | CCTCATGGCCACAAGGAT | FAM/CCAGATTGG/ZEN/CTGCAATGCCG |
| | TTG-3′ | TC/3IABkFQ/3IABkFQ/-3′ |
| | R: 5′- | |
| | TGAGGCTTCCAGGTACAA | |
| | CAG-3′ | |
| DOCK7- | F: 5′- | 5′-/56- |
| OLR1 | TAAAACAAGGGTCAATGT | FAM/AGACACAGC/ZEN/AGGATGCCAA |
| | CACTCAT-3′ | TG/3IABKFQ/-3′ |
| | R: 5′- | |
| | CAGTCTGGATCTTTAGGT | |
| | CATCA-3′ | |
| ZMPSTE24- | F: 5′- | 5′-/56- |
| ZMYM4 | GAGGAAGAAGGGAACAG | FAM/AGACACAGC/ZEN/AGGATGCCAA |
| | TGAAGA-3′ | TG/3IABKFQ/-3′ |
| | R: 5′- | |
| | CTGGAATAGGGCTCAGTA | |
| | AAAATGTTATC-3′ | |
| PTEN- | F: 5′- | 5′-/56- |
| NOLC1 | AAGCCAACCGATACTTTT | FAM/AGACACAGC/ZEN/AGGATGCCAA |
| | CTCCA-3′ | TG/3IABkFQ/-3′ |
| | R: 5′- | |
| | ATAGATGTCTAAGAGGGA | |
| | AGAGG-3′ | |
| ACTB | F: 5′- | 5′-/56- |
| | ACCCCACTTCTCTCTAAG | FAM/CCAGTCCTC/ZEN/TCCCAAGTCCA |
| | GAG-3′ | CAC/3IABkFQ/-3′ |
| | R: 5′- | |
| | GCAATGCTATCACCTCCC | |
| | CTG-3′ | |

Prediction model on fusion gene profile. Fusion gene machine learning methods were introduced to predict the recurrence status of prostate cancer. These machine learning algorithms generally take in the fusion gene status and generate a prediction probability per sample. For fusion profiling, the semi-quantitative status of each fusion gene based on Ct cycles was tabulated across all the tumor samples. The optimal Ct cycle was obtained for each fusion gene based on its differentiation between the recurrent and non-recurrent status of the samples from the UPMC cohort. Several machine learning algorithms were applied to the fusion gene profiling data, specifically: support vector machine (SVM) (15), random forest (RF) (16 and 17), linear discriminant analysis (LDA) (18), and logistic regression (19). For all these methods, leave-one-out cross-validation (LOOCV) was performed on the training cohort to evaluate the prediction algorithms and select the best parameters of 14 fusion gene combinations. The best algorithms were then applied to the whole training cohort to train a model and apply to the testing cohort. Eventually, the training and testing cohorts were pooled together to generate the best model for the prediction of recurrence based on LOOCV. All bio statistical analyses were performed by R programming and available R packages: ‘randomForest’, ‘MASS’, and ‘e1071’; R Foundation (www.r-project.org).

Prediction model integrating fusion genes, Gleason score, and serum PSA. Clinical features such as Gleason score and serum PSA were also available for the prediction of cancer recurrence. The machine-learning algorithm was first applied to these clinical features individually. With regard to Gleason score, the combined Gleason score optimal for use in predicting recurrence was selected. For serum PSA, the cutoff value that best differentiated recurrence from nonrecurrence was chosen. In order to integrate fusion gene profiling, Gleason score and serum PSA, the above machine learning models were applied to all the three features together to train the best model and generate the prediction probability for the fusion+Gleason+PSA model. If the probability was equal to or less than 0.5, it was predicted as non-recurrent. If the probability was more than 0.5, it was predicted as recurrent. Similarly, fusion gene status combined with Gleason score generated probability for fusion+Gleason models, while fusion combined with serum PSA prediction generated probability for fusion+PSA models. Similar to models only involving fusion gene data, the models integrating fusion gene, Gleason score, and serum PSA were applied to the training cohort. The best parameters selected by LOOCV were used as the final model for the training cohort and then applied to the validation cohort for evaluation. Eventually, both cohorts were pooled together to provide a final prediction model for recurrent cases. All the biostatistics analysis was performed by R programming.

### Results

### Fusion Genes Frequently Present in Prostate Cancer Samples.

The role of fusion genes in promoting the metastasis/recurrence of prostate cancer is still poorly understood. The present disclosure analyzed 14 fusion genes that were previously found to be present in the prostate cancer samples, including MAN2A1-FER, TRMT11-GRIK2, MTOR-TP53BP1, CCNH-C5orf30, KDM4B-AC011523.2, SLC45A2-AMACR, TMEM135-CCDC67, LRRC59-FLJ60017, CLTC-ETV1, PCMTD1-SNTG1, ACPP-SEC13, DOCK7-OLR1, ZMPSTE24-ZMYM4, and Pten-NOLC1. The present disclosure provides analyses of a multi-institutional cohort that includes 271 samples of radical prostatectomy with adequate clinical information from University of Pittsburgh Medical Center (UPMC), 191 from University of Wisconsin Madison, and 112 from Stanford Medical Center. Most of these samples had a clinical follow-up at least 5 years after the surgical treatment.

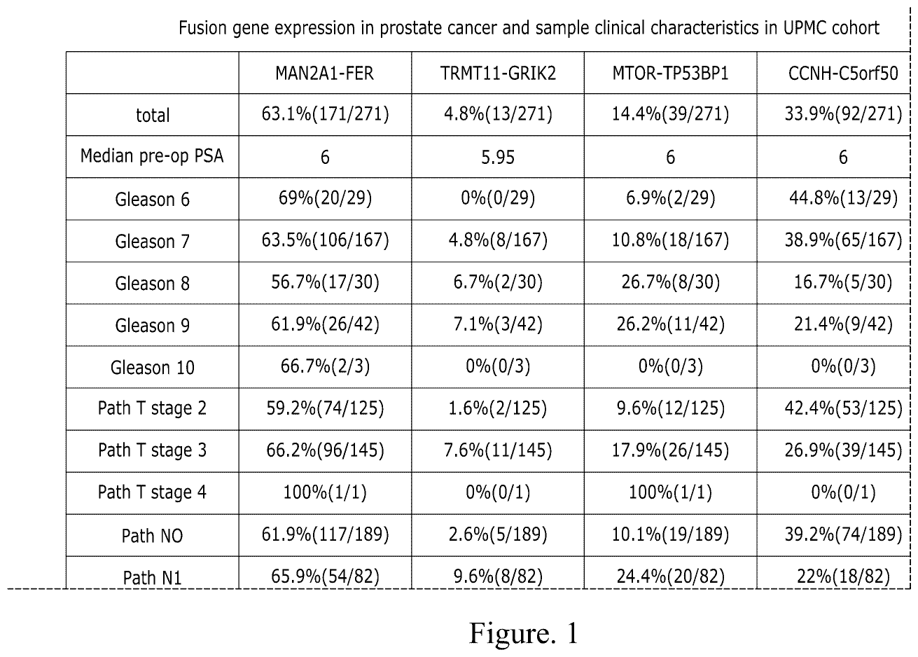

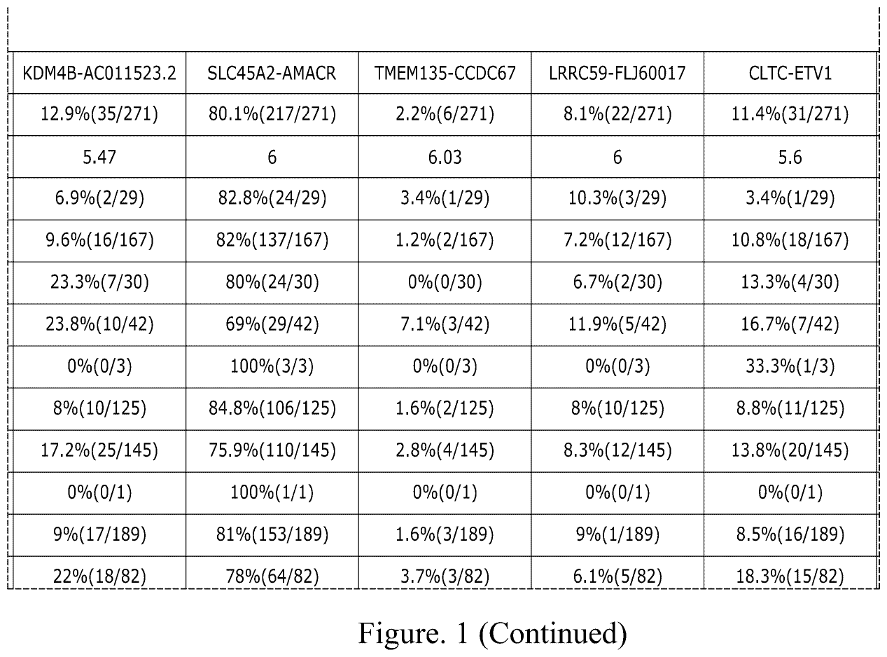

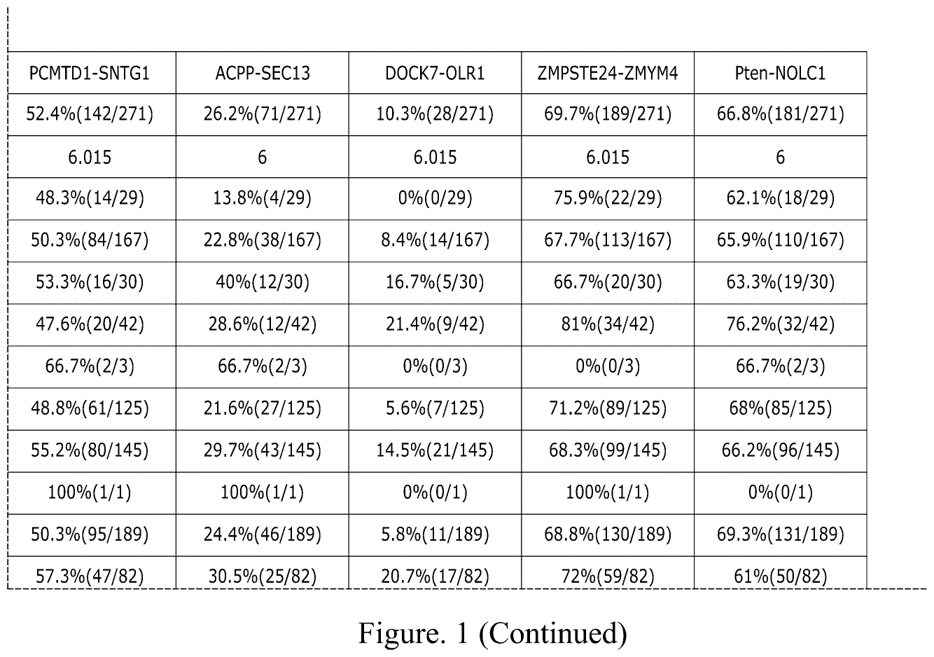

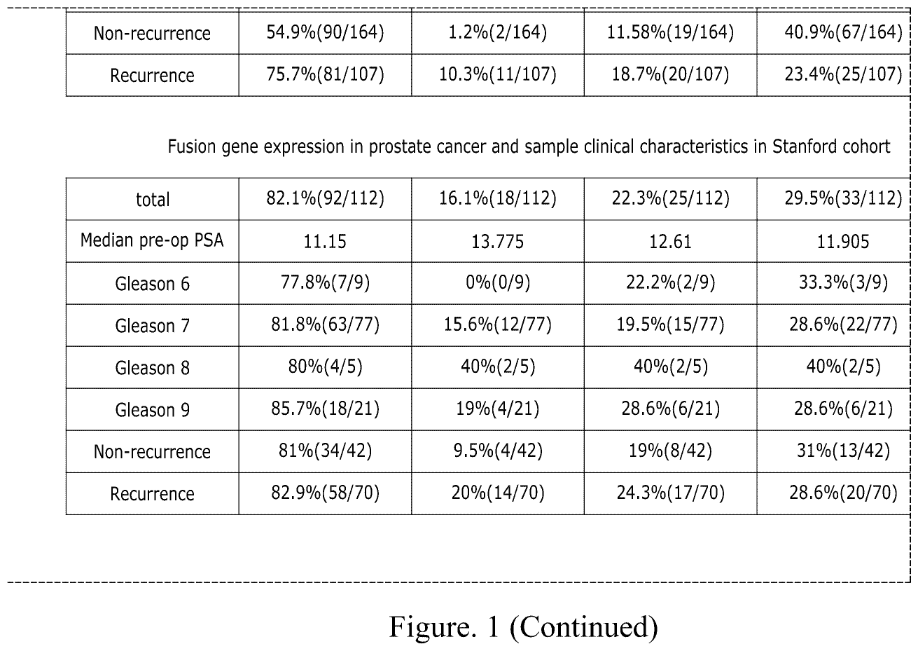

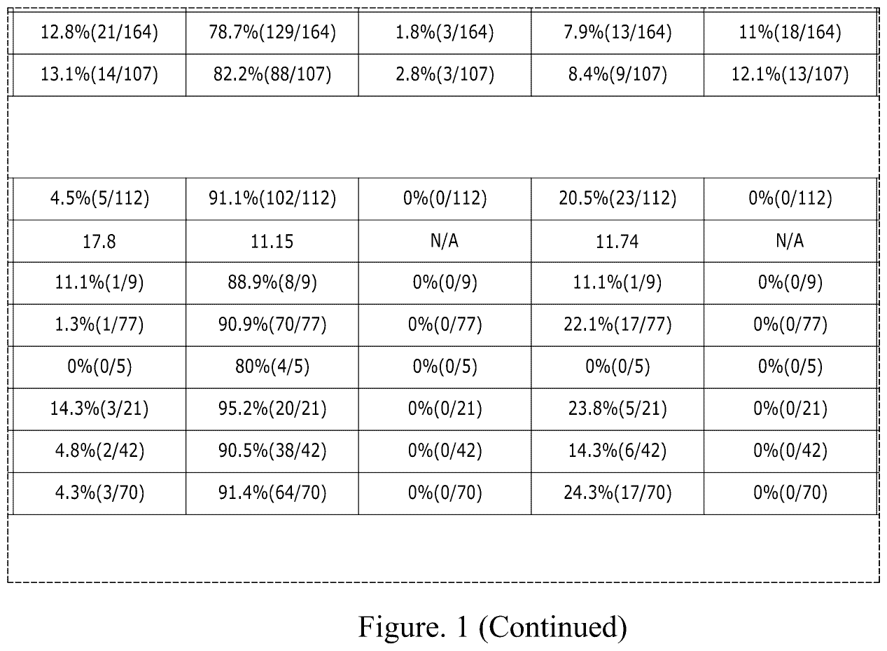

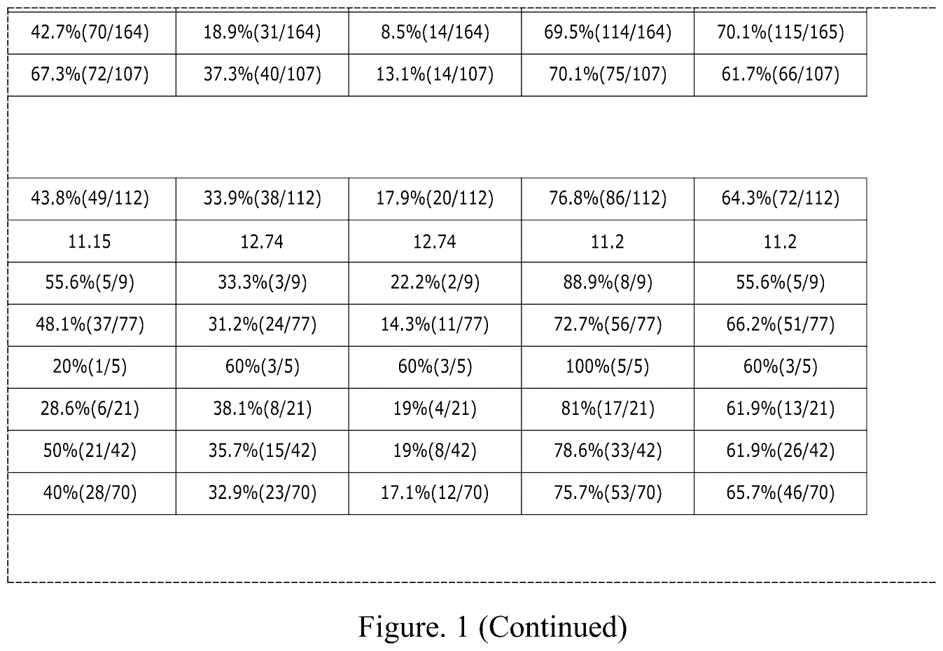

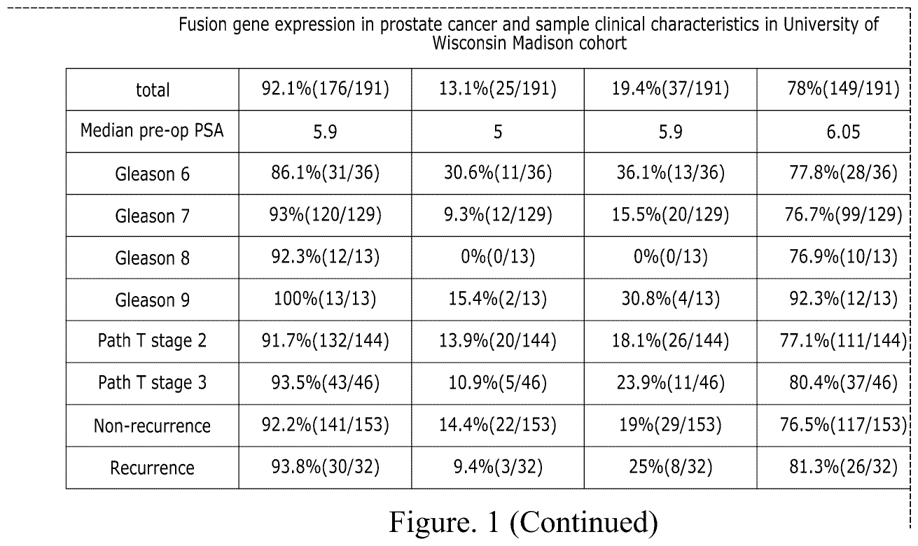

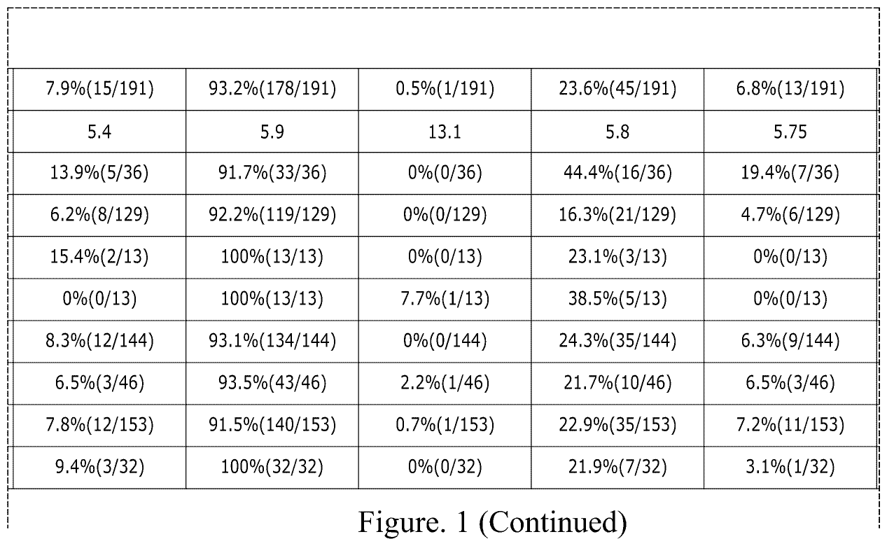

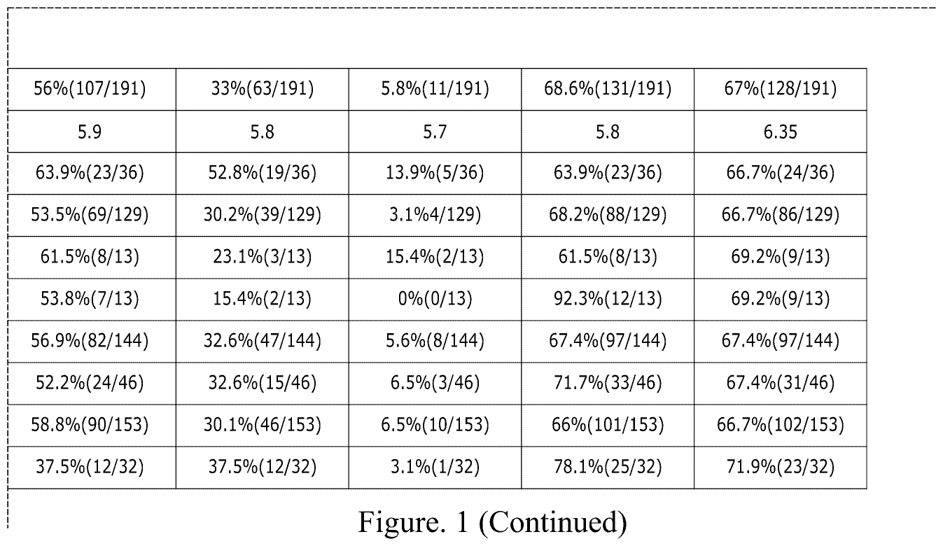

As shown in FIG. 1, all 14 fusion genes were detected in the prostate cancer samples of the combined cohorts. SLC45A2-AMACR had the highest detection rate (86.8%) of all fusion genes in the combined cohorts, ranging from 80.1% of UPMC cohort to 93.2% of Wisconsin cohort. This was followed by MAN2A1-FER (76.5%), ZMPSTE24-ZMYM4 (70.7%), and Pten-NOLC1 (66.4%), while TMEM135-CCDC67 had the lowest frequency, only 1.2% of the samples were positive for the fusion gene. In general, the frequencies of the fusion gene distribution were comparable among the three cohorts, except CCNH-C5orf30, which was detected with significantly higher frequency in the Wisconsin cohort (78% versus 29.5% or 33.9% for UPMC and Stanford cohorts, respectively).

Fusion Gene Expressions Associated with Clinical and Pathological Features of Prostate Cancer.

Association analysis in the UPMC cohort showed that the presence of mTOR-TP53BP1 (p=0.0028), KDM4B-AC011523.2 (p=0.02), ACPP-SEC13 (p=0.007), and DOCK7-OLR1 (p=0.03) in the prostate cancer samples was associated with increased risk for a high combined Gleason score (8-10), while CCNH-C5orf30 (p=0.01) was associated with a low combined Gleason score (6-7). In addition, the presence of MAN2A1-FER (p=0.046), MTOR-TP53BP1 (p=0.0018), KDM4B-AC011523.2 (p=0.025), and PCMTD1-SNTG1 (p=0.021) was associated with a high Gleason score, while CCNH-C5orf30 was associated with a low Gleason score (p=0.0027). The presence of MAN2A1-FER (p=0.01) and MTOR-TP53BP1 (p=0.007) in the prostate cancer samples was also associated with a more advanced pathological cancer stage (T3/T4), while the presence of CCNH-C5orf30 (p=0.027) was associated with cancers of the less invasive stage (T2). Strong expression of MAN2A1-FER (Ct≤35, p=0.0008) and the presence of mTOR-TP53BP1 (p=0.0007) were associated with higher pre-operative serum PSA levels. Six fusion genes were associated lymph node involvement: MAN2A1-FER (p=0.0036), TRMT11-GRIK2 (p=0.025), MTOR-TP53BP1 (p=0.0088), SLC45A2-AMACR (p=0.028), PCMTD1-SNTG1 (p=0.033), and DOCK7-OLR1 (p=0.0031). Similar to lymph node involvement, 6 fusion genes were associated with increased risk of biochemical recurrence of prostate cancer: MAN2A1-FER (p=9.4×10⁻⁶), TRMT11-GRIK2 (p=0.007), MTOR-TP53BP1 (p=4.97×10⁻⁶), PCMTD1-SNTG1 (p=0.00018), ACPP-SEC13 (p=0.0019), and DOCK7-OLR1 (p=0.0017). The presence of CCNH-C5orf30 was associated with decreased risk for the recurrence of prostate cancer (p=0.00026).

To investigate whether fusion genes were also associated with similar clinical characteristics of the prostate cancer samples in independent cohorts, association analyses were performed on Stanford and Wisconsin cohorts. The Wisconsin cohort contains 17.3% prostate cancers that were recurrent, while Stanford's had 62.5% recurrent prostate cancers. To make the analyses balanced and comparable, the Wisconsin and Stanford cohorts were combined into one external cohort with sample number and clinical characteristics similar to those from UPMC (39.5% recurrent of 271 samples). The combined cohort has a total of 303 prostate cancer samples including 297 samples containing clinical follow-up information. Thirty-four percent (102/297) of the samples of the combined cohort had known prostate cancer recurrence. Association analyses of the combined external cohort showed that the presence of MTOR-TP53BP1 (p=0.03), LRRC59-FLJ60017 (p=0.02), and CLTC-ETV1 (p=0.006) was associated with higher Gleason score. Strong expressions of MAN2A1-FER (Ct≤34 cycles, p=0.006) and Pten-NOLC1 (Ct≤33 cycles, p=0.04) were also associated with higher Gleason scores. The presence of Pten-NOLC1 (p=0.03) was associated with higher pre-operative serum PSA levels. The expressions of DOCK7-OLR1 (p=0.04) and of ZMPSTE24-ZMYM4 (p=0.04) were associated lower PSA-free survival. In contrast, good expression of CCNH-C5orf30 (Ct ≤37) was associated with a lower Gleason score (p=0.005), lower PSA level (p=4.1×10⁻⁵), a lower recurrent rate (p=0.0006), and better PSA-free survival (p=0.0002).

### Fusion Gene-Based Machine Learning Models to Predict Prostate Cancer Recurrence in UPMC Cohort.

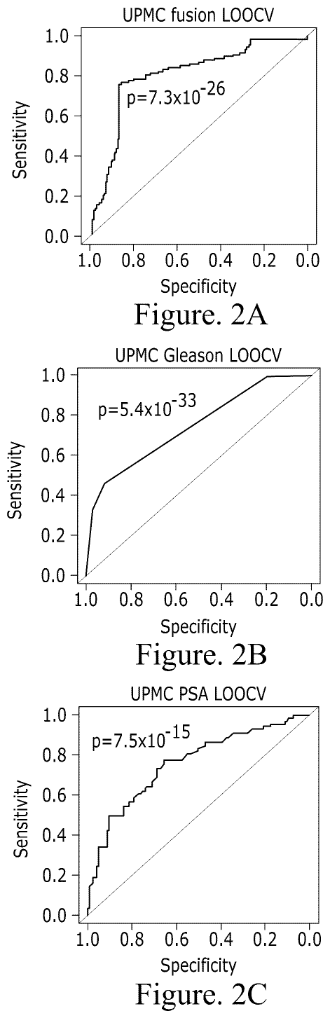

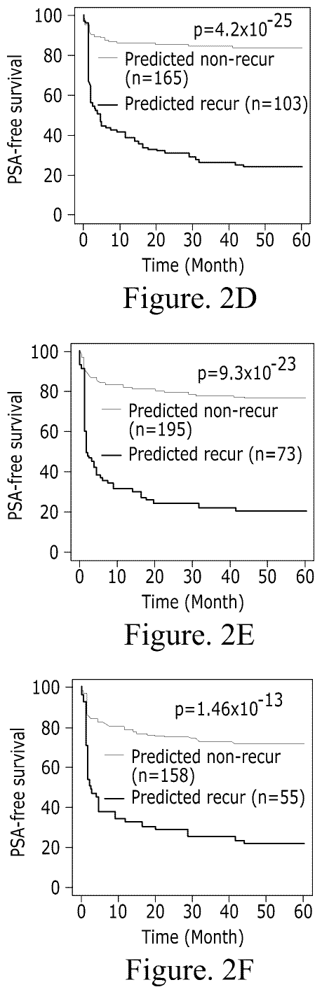

To investigate whether individual fusion gene or the combination of fusion genes were predictive of prostate cancer recurrence outcomes, multiple machine learning models utilizing various combinations of fusions with the optimal intensity cutoffs were employed to analyze the UPMC prostate cancer cohort based on “leave-one out cross-validation” (LOOCV) method. A total of 764 models were constructed, Of which 457 models had prediction rates above 70% (FIG. 1). The support vector machine (SVM) model by combining 6 fusion genes [MAN2A-FER (Ct≤34), TRMT11-GRIK2 (Ct≤43), MTOR-TP53BP1(Ct≤42), CCNH-C5orf30 (negative), PCMTD1-SNTG1 (Ct≤38), and ACPP-SEC13 (Ct≤40)] produced an accuracy of 81.9%, with sensitivity of 76.6%, and specificity of 85.4%. The model also generated a Youden index of 0.62 (FIG. 2). The PSA-free survival analysis of the 6-fusions SVM model showed that 24.3% patients survived 5 years PSA-free if the cancer was predicted as recurrent, while 85% patients had no recurrence for at least 5 years if predicted as non-recurrent (FIG. 2, p=4.2×10⁻²).

### Incorporation of Fusion Gene Detection Enhanced Gleason Score Prediction of Prostate Cancer Recurrence in the UPMC Cohort.

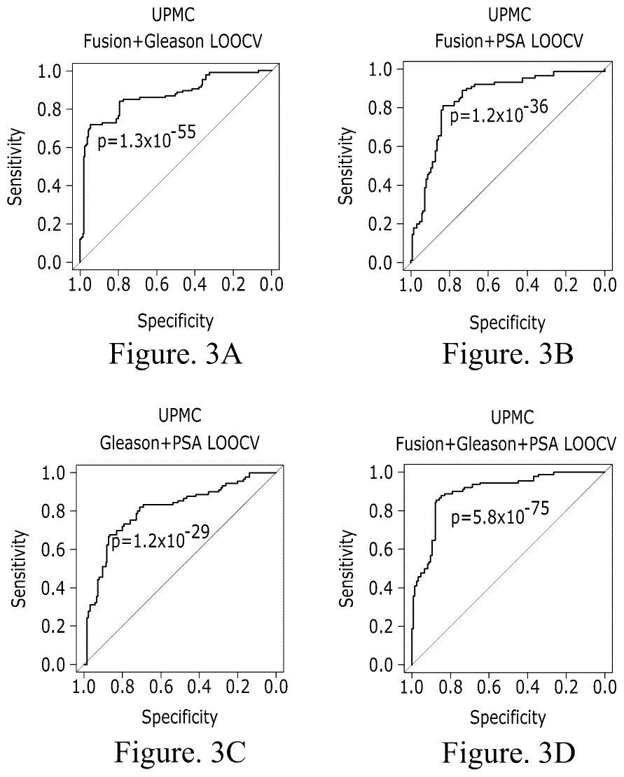

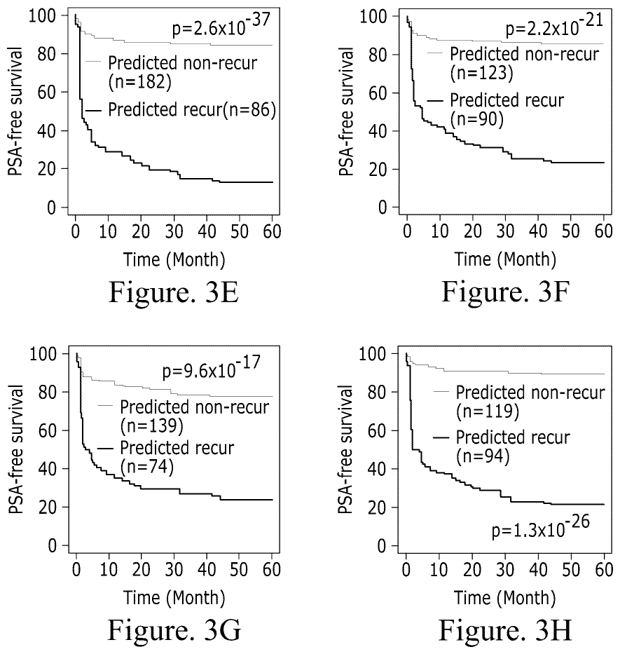

The prediction analysis based on Gleason scores showed that the cutoff of Gleason score at 8 in the UPMC cohort has the best prediction: 77.9% accuracy with sensitivity of 57% and specificity of 91.5% (FIG. 2 and Table 2). To investigate whether the combination of fusion genes and Gleason scores enhanced the prediction of prostate cancer recurrence, Gleason score was incorporated into the machine learning LOOCV analysis. A total of 442 models of different combinations showed an accuracy above 80% when fusion gene profiling was combined with Gleason score. As shown in FIG. 3, a support vector machine model using the detection of six fusions [MAN2A-FER (Ct≤34), TRMT11-GRIK2 (Ct≤43), MTOR-TP53BP1(Ct≤42), CCNH-C5orf30 (negative), PCMTD1-SNTG1 (Ct≤38), and ACPP-SEC13 (Ct≤40)] +Gleason score accurately predicted prostate cancer recurrence in 85.2% of cases, with a sensitivity of 72% and specificity of 94%. The survival analysis showed that only 12.8% of patients had recurrence-free survival for 5 years after surgery if the cancer was predicted as recurrent. In contrast, 84.6% of patients had recurrence-free survival of 5 years after surgery if the cancer was predicted as non-recurrent. These results represented an improvement over the Gleason score alone: 20.5% having recurrence-free survival for 5 years after the surgery if Gleason score was 8 or above, and 76.9% having no recurrence if Gleason score was 7 or less (FIGS. 2 and 3).

TABLE 2
Gleason prediction UPMC

| Feature | AUC | Accuracy | Sensitivity | Specificity | YoudenIndex | TP | FP | TN | FN |
|---|---|---|---|---|---|---|---|---|---|
| Gleason >= 5 | 0.786016 | 0.394834 | 1 | 0 | 0 | 107 | 164 | 0 | 0 |
| Gleason >= 6 | 0.786016 | 0.394834 | 1 | 0 | 0 | 107 | 164 | 0 | 0 |
| Gleason >= 7 | 0.786016 | 0.501845 | 1 | 0.176829 | 0.176829 | 107 | 135 | 29 | 0 |
| Gleason >= 8 | 0.786016 | 0.778598 | 0.570093 | 0.914634 | 0.484728 | 61 | 14 | 150 | 46 |
| Gleason >= 9 | 0.786016 | 0.726937 | 0.364486 | 0.963415 | 0.327901 | 39 | 6 | 158 | 68 |
| Gleason = 10 | 0.786016 | 0.616236 | 0.028037 | 1 | 0.028037 | 3 | 0 | 164 | 104 |

TP: true positive; FP: false positive; TN: true negative; FN: false negative

### Fusion Gene Detection Improved PSA Prediction of Prostate Cancer Recurrence in the UPMC Cohort.

The use of Serum PSA alone was moderately effective in predicting the recurrence of prostate cancer. A high serum PSA level was correlated with the risk of prostate cancer recurrence. Indeed, a PSA level above 9.77 ng/ml correctly predicted 73.5% of cases of prostate cancer recurrence in the UPMC cohort, with a sensitivity of 50% and specificity of 90.4% (Table 3 and FIG. 2). When fusion gene profiling was combined with the PSA prediction analysis, 265 models of different combinations showed prediction accuracy rates above 75%. The top prediction model was a SVM model that incorporated serum PSA level cutoff of 9.77 ng/ml with the presence of five fusion genes (FIG. 3): MAN2A-FER (Ct≤34), MTOR-TP53BP1 (Ct≤42), CCNH-C5orf30 (negative), PCMTD1-SNTG1 (Ct≤38), and ACPP-SEC13 (Ct≤40). The model produced 82.3% accuracy with 80% sensitivity and 84% specificity. Survival analyses showed that 23.3% of patients survived 5 years PSA-free if the cancer was predicted as recurrent, while 85.4% patients survived 5 years PSA-free if the cancer was predicted as non-recurrent (FIG. 3, p=2.2×10⁻²¹). This finding represented a moderate improvement over the use of PSA used alone: 21.8% PSA-free survival for 5 years if PSA above 9.77 ng/ml and 72.2% PSA-free survival if PSA below 9.77 ng/ml (FIG. 2, p=1.5×10⁻¹³) (FIG. 2).

TABLE 3
Serum PSA prediction UPMC

| Feature | AUC | Accuracy | Sensitivity | Specificity | YoudenIndex | TP | FP | TN | FN |
|---|---|---|---|---|---|---|---|---|---|
| PSA >= 9.77 | 0.7604 | 0.735 | 0.5 | 0.904 | 0.404 | 45 | 12 | 113 | 45 |
| PSA >= 5.84 | 0.7604 | 0.707 | 0.7778 | 0.656 | 0.434 | 70 | 43 | 82 | 20 |

TP: true positive; FP: false positive; TN: true negative; FN: false negative

### Combination of Fusion Gene, Serum PSA and Gleason Score Predicting the Recurrence of Prostate Cancer in the UPMC Cohort.

To investigate whether combination of serum PSA, Gleason score and fusion genes improves the prostate cancer recurrence further, 385 models with various combinations based on the best intensity cutoffs using LOOCV were constructed. A total of 317 models yielded prediction accuracy rates of 80% or better. The Random Forest (RF) model, which combined Gleason score, serum PSA, and three fusion genes [MAN2A-FER (Ct≤34), CCNH-C5orf30 (negative), DOCK7-OLR1 Ct≤41)] produced the highest Youden index, with 84.7% accuracy, 84.4% sensitivity and 84.8% specificity (FIG. 3). These results represent an improvement over the use of Gleason score plus serum PSA: 78.6% accuracy, with 64.4% sensitivity and 88.8% specificity (Table 4 and FIG. 3). Survival analyses showed that 21.3% of prostate cancer patients survived 5 years PSA-free after surgery if the cancer was predicted as recurrent by the RF model, while 89.1% of patients experienced no recurrence for 5 years after surgery if the cancer was predicted as non-recurrent (FIG. 3, p=1.3×10⁻²⁶. On the other hand, the best Gleason score plus serum PSA model (Logistic) generated a 21.1% PSA-free survival for 5 years if the cancer was predicted as recurrent and 78.2% PSA-free survival if the cancer was predicted as non-recurrent (FIG. 3, p=9.6×10⁻¹⁷).

TABLE 4
Gleason + PSA UPMC

| Method | AUC | Accuracy | Sensitivity | Specificity | YoudenIndex | TP | FP | TN | FN |
|---|---|---|---|---|---|---|---|---|---|
| LDA | 0.813867 | 0.776744 | 0.6 | 0.904 | 0.504 | 54 | 12 | 113 | 36 |
| SVM | 0.706489 | 0.776744 | 0.633333 | 0.88 | 0.513333 | 57 | 15 | 110 | 33 |
| RF | 0.804711 | 0.781395 | 0.655556 | 0.872 | 0.527556 | 59 | 16 | 109 | 31 |
| Logistic | 0.828 | 0.786047 | 0.644444 | 0.888 | 0.532444 | 58 | 14 | 111 | 32 |

TP: true positive; FP: false positive; TN: true negative; FN: false negative

### Stanford/Wisconsin Cohort Validation of the Fusion Gene Enhancement of Prediction of Prostate Cancer Recurrence.

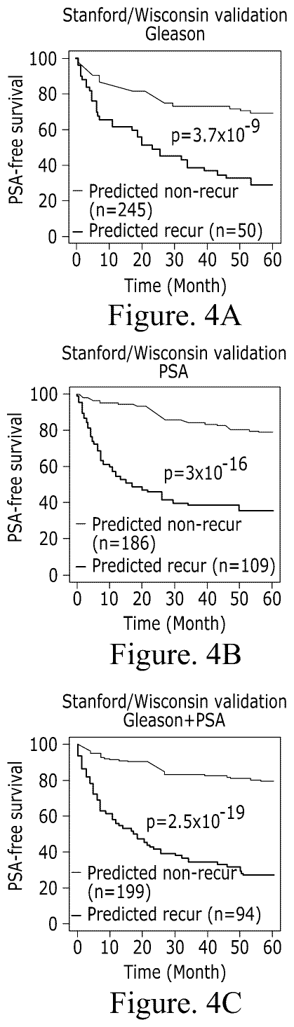

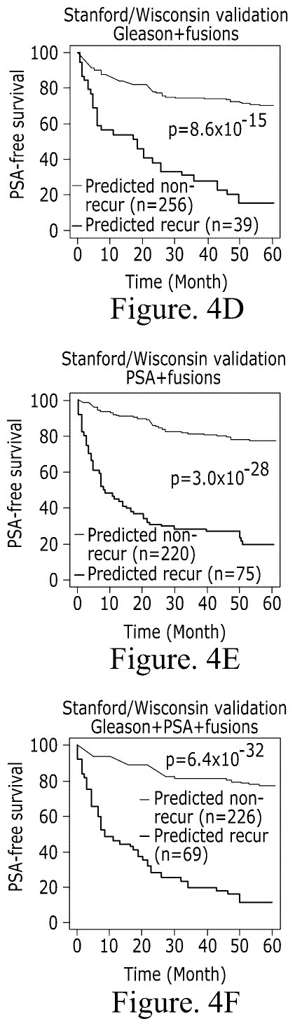

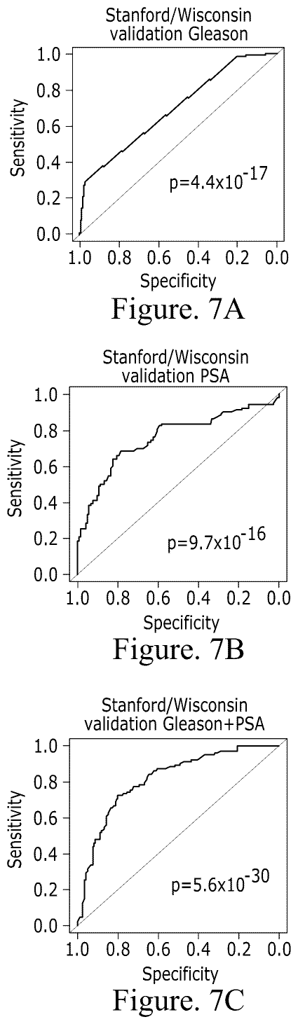

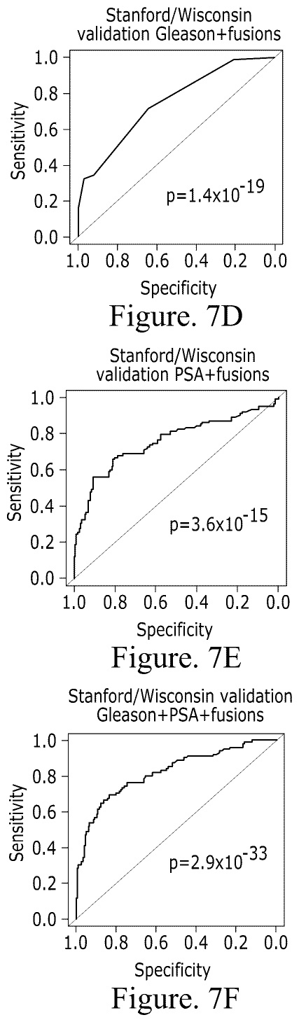

The present disclosure shows that 764 machine learning models trained from the UPMC cohort were applied to the Stanford/Wisconsin cohort. However, none of these models had a prediction rate reaching 70%. The optimized cutoff of Gleason score based on data from the UPMC cohort was then applied to predict the outcomes of prostate cancer patients from Stanford/Wisconsin cohort (combined Gleason>8 as recurrent). The results produced a Youden index of 0.27 and yielded 72.4% accuracy, with 34.3% sensitivity and 92.3% specificity (Table 5, FIG. 7, p=4.4×10⁻¹⁷). To investigate whether fusion gene detection enhanced the prediction of prostate cancer recurrence by Gleason score, the 764 model algorithms developed from the UPMC cohort were applied to the Stanford/Wisconsin cohort for cross-validation, with 52 models yielded prediction accuracy rates exceeding 72.5%. One of the models was a Linear Discriminatory Analysis (LDA) model that integrated two fusion genes [TRMT11-GRIK2 (Ct≤43) and CCNH-C5orf30 (negative)] with Gleason score, which yielded the highest Youden index at 0.3, and a prediction accuracy of 75%, with 32.3% sensitivity and 96.9% specificity (FIG. 7). The same model also predicted 79% accuracy for the UPMC cohort. Survival analysis showed that 70.6% of patients survived 5-years without the recurrence of the prostate cancer when predicted as non-recurrent, while only 15.4% of patients survived similar periods when predicted as recurrent by the model (FIG. 4, p=8.7×10⁻¹⁵). This represented a moderate improvement over Gleason score alone: 70.2% survived 5 years without recurrence if Gleason score was 7 or lower, while 28.7% survived a similar period without recurrence if Gleason score was 8 or above (FIG. 4, p=3.7×10⁻⁹).

TABLE 5
Gleason validation Stanford/Wisconsin

| Feature | AUC | Accuracy | Sensitivity | Specificity | YoudenIndex | TP | FP | TN | FN |
|---|---|---|---|---|---|---|---|---|---|
| Gleason >= 5 | 0.705053 | 0.343434 | 1 | 0 | 0 | 102 | 195 | 0 | 0 |
| Gleason >= 6 | 0.705053 | 0.343434 | 1 | 0 | 0 | 102 | 195 | 0 | 0 |
| Gleason >= 7 | 0.705053 | 0.478114 | 0.990196 | 0.210256 | 0.200452 | 101 | 154 | 41 | 1 |
| Gleason >= 8 | 0.705053 | 0.723906 | 0.343137 | 0.923077 | 0.266214 | 35 | 15 | 180 | 67 |
| Gleason >= 9 | 0.705053 | 0.740741 | 0.284314 | 0.979487 | 0.263801 | 29 | 4 | 191 | 73 |
| Gleason = 10 | 0.705053 | 0.656566 | 0 | 1 | 0 | 0 | 0 | 195 | 102 |

TP: true positive; PP: false positive; TN: true negative; FN: false negative

PSA was used as the sole criteria to predict prostate cancer recurrence in Stanford/Wisconsin cohort based on the training data from the UPMC cohort yielded 74.7% accuracy with 67.6% sensitivity and 78.5% specificity Table 6 and FIG. 7). Among 56 models of fusion gene profiling plus serum PSA, the prediction accuracy rate exceeded 75%. A three fusion gene [TRMT11-GRIK2 (Ct≤43), CCNH-C5orf30 (negative), and ACPP-SEC13 (Ct≤40)] logistic model integrated with PSA yielded a 78.9% accuracy with 56% sensitivity and 90.8% specificity (FIG. 7). The same model predicted 80% of recurrence correctly in the UPMC cohort. Survival analysis showed that 77% of patients survived 5 years without cancer recurrence when the cancer was predicted as non-recurrent, while only 17.8% of patients have no recurrence if the cancer was predicted as recurrent (FIG. 4, p=3.0×10⁻²⁸). These findings represent a moderate improvement over the survival prediction by PSA alone: 78.8% of patients survived 5 years without recurrence when PSA was less than 9.77 ng/ml, while 38.8% of patients survived the same period without recurrence when PSA was more than 9.77 ng/ml (FIG. 4, p=3×10⁻¹⁶)

TABLE 6
PSA validation Stanford/Wisconsin

| Feature | AUC | Accuracy | Sensitivity | Specificity | YoudenIndex | TP | FP | TN | FN |
|---|---|---|---|---|---|---|---|---|---|
| PSA >= 9.77 | 0.757215 | 0.747475 | 0.676471 | 0.784615 | 0.461086 | 69 | 42 | 153 | 33 |
| PSA >= 5.84 | 0.757215 | 0.602694 | 0.833333 | 0.482051 | 0.315385 | 85 | 101 | 94 | 17 |

TP: true positive; FP: false positive; TN: true negative; FN: false negative

When combining serum PSA and Gleason score, the prediction of prostate cancer recurrence improved to 76.8%, with Youden index 0.45 (Table 7), better than either PSA or Gleason score alone. To investigate whether the integration of fusion genes, PSA level, and Gleason score further improve the prediction rate, 764 algorithms developed from UPMC training cohort were applied to the Stanford/Wisconsin cohort for validation analyses. Seventy-three algorithms produced an accuracy exceeding 77%. Among them, a LDA model integrating four fusion genes [TRMT11-GRIK2 (Ct≤43), CCNH-C5orf30 (negative), ACPP-SEC13 (Ct≤40), and DOCK7-OLR1 Ct≤41)] with Gleason score and serum PSA level generated a prediction accuracy of 79.5% with 53.9% sensitivity, 92.8% specificity and 0.47 Youden index (FIG. 7). The same model predicted 82.3% prediction accuracy in the UPMC cohort. Survival analyses showed that 78% of patients survived 5 years without recurrence if the cancer was predicted as non-recurrent by using the four fusion genes+Gleason+PSA LDA model, while only 11.6% of patients experienced no recurrence in the same period if the cancer was predicted as recurrent (FIG. 4, p=6.4×10⁻³²). These results represent an improvement on the optimal Gleason+PSA model: 78% of patients survived 5 years without recurrence if the cancer was predicted as non-recurrent, while 26.7% of patients experienced no recurrence if the cancer was predicted as recurrent (FIG. 4, p=2.5×10⁻¹⁹). In general, these results indicate that the addition of the fusion gene algorithm improved the prediction accuracy rate of PSA and/or Gleason score on prostate cancer recurrence in two independent cohorts.

TABLE 7
Gleason + PSA validation Stanford/Wisconsin

| Feature | Method | AUC | Accuracy | Sensitivity | Specificity | YoudenIndex | TP | FP | TN | FN |
|---|---|---|---|---|---|---|---|---|---|---|
| gleason + PSA | Logistic | 0.8171 | 0.7677 | 0.5882 | 0.8615 | 0.4498 | 60 | 27 | 168 | 42 |

TP: true positive; FP: false positive; TN: true negative; FN: false negative

### Combining UPMC, Stanford, and University of Wisconsin Cohorts for Cross-Validation Prediction

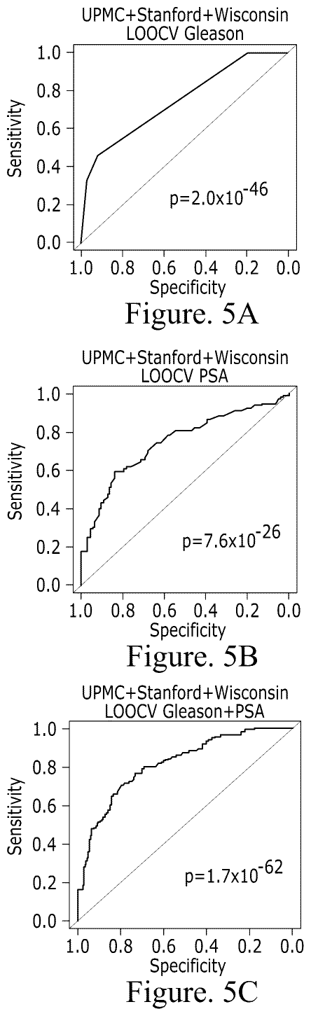

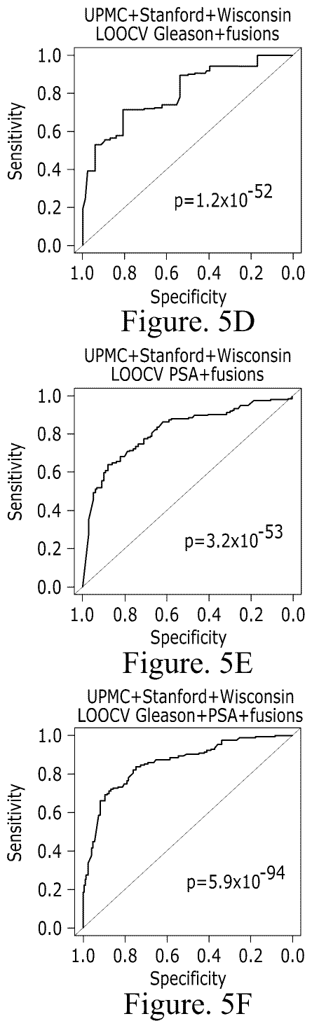

When combined all the cohorts together (574 cases), Gleason score alone (cutoff=8, optimal) yielded 75% accuracy (Table 8). The present disclosure found that most (440) of the fusion gene-containing algorithms combined with Gleason score exceed 76% based on LOOCV. One of the models called five fusion genes [MAN2A-FER (Ct≤34), TRMT11-GRIK2 (Ct≤43), MTOR-TP53BP1(Ct≤42), CCNH-C5orf30 (negative), and ACPP-SEC13 (Ct≤40)] Random Forest improved the Gleason prediction accuracy from 75% to 78% (FIG. 5). Serum PSA also yielded a prediction of 74.2% when using the optimal cutoff (9.77 ng/ml, Table 9 and FIG. 5). When serum PSA was incorporated into an RF model that used the detection of five fusion genes [MAN2A-FER (Ct≤34), MTOR-TP53BP1(Ct≤42), CCNH-C5orf30 (negative), CLTC-ETV1(Ct≤37), and ACPP-SEC13 (Ct≤40)], the accuracy of predicting prostate cancer recurrence improved to 78.7% (FIG. 5). When Gleason score, serum PSA and a five fusion gene [MAN2A-FER (Ct≤34), TRMT11-GRIK2 (Ct≤43), MTOR-TP53BP1(Ct≤42), CCNH-C5orf30 (negative), and ACPP-SEC13 (Ct≤40)] were combined into a RF model, the prediction accuracy improved to 82.4%, with 68.8% sensitivity, 90.6% specificity and 0.59 Youden index (FIG. 5). These results represented an improvement in prediction accuracy over the use of combined serum PSA with Gleason score: 77.1% accuracy, 55.8 sensitivity, 90% specificity and 0.46 Youden index (FIG. 5).

TABLE 8
Gleason prediction UPMC + Stanford + Wisconsin

| Feature | AUC | Accuracy | Sensitivity | Specificity | YoudenIndex | TP | FP | TN | FN |
|---|---|---|---|---|---|---|---|---|---|
| Gleason >= 5 | 0.746731 | 0.367958 | 1 | 0 | 0 | 209 | 359 | 0 | 0 |
| Gleason >= 6 | 0.746731 | 0.367958 | 1 | 0 | 0 | 209 | 359 | 0 | 0 |
| Gleason >= 7 | 0.746731 | 0.489437 | 0.995215 | 0.194986 | 0.190201 | 208 | 289 | 70 | 1 |
| Gleason >= 8 | 0.746731 | 0.75 | 0.45933 | 0.91922 | 0.37855 | 96 | 29 | 330 | 113 |
| Gleason >= 9 | 0.746731 | 0.734155 | 0.325359 | 0.972145 | 0.297504 | 68 | 10 | 349 | 141 |
| Gleason = 10 | 0.746731 | 0.637324 | 0.014354 | 1 | 0.014354 | 3 | 0 | 359 | 206 |

TABLE 8
PSA prediction UPMC + Stanford + Wisconsin

| Feature | Method | AUC | Accuracy | Sensitivity | Specificity | YoudenIndex | TP | FP | TN | FN |
|---|---|---|---|---|---|---|---|---|---|---|
| PSA >= 9.77 | PSA | 0.74541 | 0.742188 | 0.59375 | 0.83125 | 0.425 | 114 | 54 | 266 | 78 |

TP: true positive; FP: false positive; TN: true negative; FN: false negative

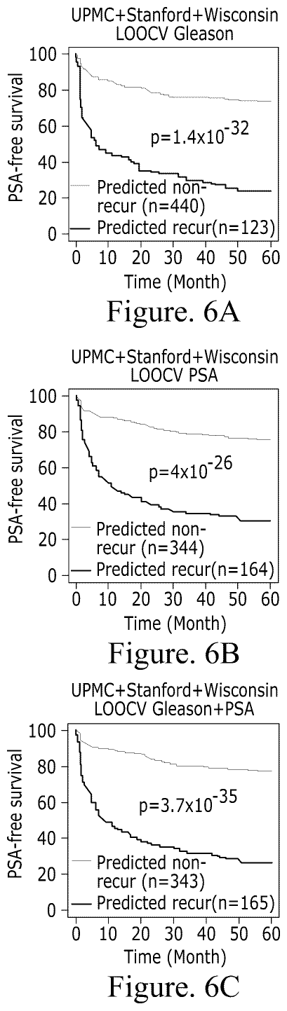

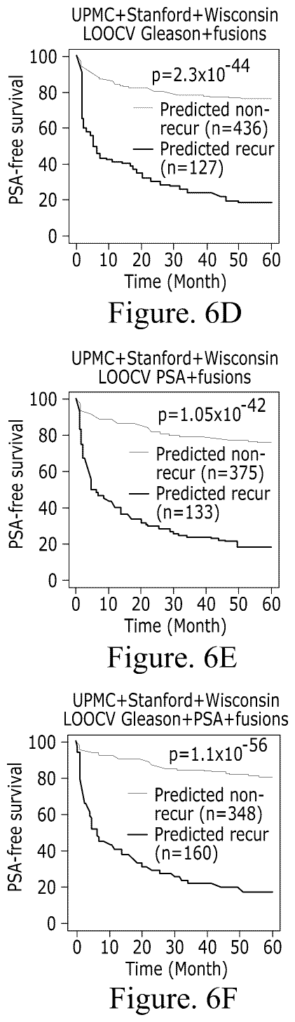

The survival analysis showed that 76% of patients survived 5 years recurrence-free if the cancer was predicted as non-recurrent by the Fusion+Gleason RF model, while 18.4% of patients were prostate cancer-free if the cancer was predicted as recurrent by the same model (FIG. 6, p=2.3×10⁻⁴⁴). This combination yielded an improvement over Gleason score alone: 73.8% PSA-free survival if Gleason was 7 or lower and 23.9% PSA-free survival if Gleason 8 or above (p=1.4×10⁻³²). When PSA and fusion algorithms were combined, 76.5% of patients were prostate cancer-free for 5 years if the cancer was predicted negative for recurrence and 17.9% of patients were prostate cancer free for 5 years if the cancer was predicted as positive for recurrence (FIG. 6, p=1.05×10⁻²). These results compared favorably against the prediction by PSA alone: 76% of patients survived 5 years recurrence-free if serum PSA was less than 9.77 ng/ml, and 30.5% of patients had cancer recurrence in 5 years if serum PSA was above 9.77 ng/ml. When fusion gene profiling, Gleason score and PSA algorithms were combined, the prediction results were further improved: 81.9% of patients were prostate cancer recurrence-free for 5 years after the surgery if the cancer was predicted as non-recurrent by the Fusion+Gleason+PSA RF model, while only 17.2% patients were cancer recurrence-free if the cancer was predicted as recurrent by the same model (FIG. 6, p=1.1×10⁻⁵⁶). On the other hand, the results from Gleason+PSA logistic model showed that 78.3% of patients had no cancer recurrence for 5 years if the cancer was predicted as non-recurrent by the model, and 26.2% of patients had no cancer recurrence for 5 years if the cancer was predicated as recurrent (p=5.7×10⁻³⁵).

### Learning Models Most Consistent Among Cohorts

The models that worked well in the Stanford/Wisconsin validation appeared the most consistent models for clinical application: the LDA model integrating Gleason score, serum PSA level, and the detection of four fusion genes [TRMT11-GRIK2 (CT≤43), CCNH-C5orf30 (negative), ACPP-SEC13 (CT≤40), and DOCK7-OLR1 (CT≤41)] yielded 79.5% accuracy in the Stanford/Wisconsin cohort, 82.3% in the UPMC cohort, and 81.8% in the combined UPMC/Stanford/Wisconsin cohorts. Similarly, the LDA model that integrated Gleason score with the detection of two fusion genes [TRMT11-GRIK2 (CT≤43) and CCNHC5orf30 (negative)] yielded 75% accuracy in the Stanford/Wisconsin cohort, 79% in the UPMC cohort, and 77.8% in the combined UPMC/Stanford/Wisconsin cohort. When only serum PSA was available, the logistic model using PSA integrated with the detection of three fusion genes [TRMT11-GRIK2 (CT≤43), CCNH-C5orf30 (negative), and ACPP-SEC13 (CT≤40)] yielded 78.9% accuracy in the Stanford/Wisconsin cohort, 80% in the UPMC cohort, and 76% in the RF combined UPMC/Stanford/Wisconsin cohort.

### Discussion

The prediction of the clinical course of prostate cancer remains challenging. Most cases of organ-confined prostate cancer are curable by radical prostatectomy or radiation therapy. Only a fraction of prostate cancer patients experience recurrent cancer and died from the disease. Gleason score and serum PSA level had been widely used as the basis for predicting clinical outcomes of prostate cancer patients. The present disclosure showed that fusion gene models were important contributing factors in predicting the recurrence of prostate cancer. The enhancement of PSA and/or Gleason grading by fusion gene status was quite robust: several hundred combinations of fusion genes in different algorithmic models improved the accuracy over predicting prostate cancer recurrence by Gleason score, serum PSA or the combination of both. This enhancement appeared in different cohorts with highly variable clinical characteristics. The wide variety of models that improved prediction may also be useful in overcoming the heterogeneity issue of the cancer samples in which different fusion gene patterns may appear in different loci. Thus, the machine learning models described in the present disclosure can be applied to the clinical setting readily. These machine learning models can be utilized in several scenarios: when a patient has a biopsy diagnosed as prostate cancer with a Gleason score and a recent serum PSA level, the fusion gene+Gleason+PSA models may help to predict the risk of prostate cancer recurrence with the accuracies ranging from 79.5% to 84.7%. If serum PSA is not available, the fusion gene+Gleason model can be useful in predicting the recurrence of prostate cancer, with an accuracy of 74%-85.2%. In the absence of a Gleason score, the fusion gene profiling+PSA models yielded a prediction accuracy from 78.9% to 82.3%. When a patient already has radical prostatectomy, these models will help to determine whether additional adjuvant therapy is needed. It is also possible to combine these fusion gene prediction models with other methods, such as Prostate Imaging Reporting and Data System (20) or prostate genome decipher classifier (21), to improve the prediction further.

Overfitting is some of the potential problems associated with machine learning methods. Indeed, significant variations in both clinical features and fusion gene detections were present among cases from UPMC, Stanford, and University of Wisconsin cohorts. Despite these variations, fusion gene profiling consistently improved the accuracy of predicting prostate cancer recurrence in all the cohorts. Some fusion genes were consistently associated with clinical features in both the UPMC and Stanford/Wisconsin cohorts: the presence of mTOR-TP53BP1 and strong expression of MAN2A1-FER (Ct≤34) were associated with higher Gleason scores in both cohorts. The expression of DOCK7-OLR1 was associated with prostate cancer recurrence. The opposite was true for CCNH-C5orf30. In fact, the presence of CCNH-C5orf30 signaled lower Gleason score, lower cancer recurrence, and better PSA-free survival in all the cohorts. CCNH-C5orf30 fusion features a truncated cyclin H protein and an intact independent C5orf30. Cyclin H protein (CCNH) is an important regulator for cell cycle progression to mitosis (22 and 23), and basal RNA transcription (24). The truncated cyclin H from the gene fusion lacks H5′ and HC domain, and is defective in binding cdk7 protein (25). Such defects may prevent the CCNH protein from promoting cell mitosis and RNA transcription. The truncated CCNH protein due to the gene fusion may have a negative impact on the prostate cancer progression.

The present disclosure demonstrated a new tool for predicting prostate cancer clinical outcomes in patients with prostate cancer. In comparison with Gleason score and PSA, fusion gene profiling has added value for clinical patient management, because some of the gene fusions are important molecular processes to generate prostate cancer. These fusion genes are readily detectable in the blood samples of prostate cancer patients. Thus, it is possible to build similar prediction models based on the fusion gene status of blood/serum samples from prostate cancer patients. Some of these fusion genes are proven cancer drivers (1, 8, and 9), while some others are functional knockout of tumor suppressors (14). Thus, the detection of the fusion gene provides new mechanistic insight into prostate cancer progression. For patients who are positive for MAN2A1-FER, the fusion gene sensitizes the cancer cells to crizotinib and canertinib because of the ectopic tyrosine kinase activity of the fusion protein (9). The cancer cells positive for Pten-NOLC1 are sensitive to Cyclopropanecarboxylic acid-(3-(6-(3-trifluoromethyl-phenylamino)-pyrimidin-4-ylamino)-phenyl)-amide, a potent EGFR inhibitor because Pten-NOLC1 promotes the expression of EGFR and its downstream signaling molecules (1), while cancer cells positive for SLC45A2-AMACR are sensitive to SCH772984, an inhibitor for ERK, due to the direct activation of ERK2 by the translocated AMACR protein (8). The cancer cells harboring any of these gene fusions will be targetable by gene-editing technology through the insertion of a suicide gene into the breakpoints of their recombinant genome (26). Thus, the incorporation of fusion gene detection into the prostate cancer diagnostic scheme benefits the patients in diagnosis, prognosis, cancer progression surveillance, and treatment.

### CONCLUSION

The methods provided herein demonstrate the development of a machine learning approach for determining whether a subject is at risk of prostate cancer recurrence, which integrates the fusion gene status of a patient in combination with Gleason score or serum PSA level, or both, thereby providing enhanced prediction capabilities.

### REFERENCES

1. Luo J H, Liu S, Tao J, Ren B G, Luo K, Chen Z H, et al. Pten-NOLC1 fusion promotes cancers involving MET and EGFR signalings. Oncogene 2021; 40(6):1064-76 doi 10.1038/s41388-020-01582-8.
2. Yu Y P, Ding Y, Chen Z, Liu S, Michalopoulos A, Chen R, et al. Novel fusion transcripts associate with progressive prostate cancer. The American journal of pathology 2014; 184(10):2840-9.
3. Luo J H, Liu S, Zuo Z H, Chen R, Tseng G C, Yu Y P. Discovery and Classification of Fusion Transcripts in Prostate Cancer and Normal Prostate Tissue. The American journal of pathology 2015.
4. Yu Y P, Liu S, Huo Z, Martin A, Nelson J B, Tseng G C, et al. Genomic Copy Number Variations in the Genomes of Leukocytes Predict Prostate Cancer Clinical Outcomes. PloS one 2015; 10(8):e0135982.
5. Yu Y P, Song C, Tseng G, Ren B G, Laframboise W, Michalopoulos G, et al. Genome abnormalities precede prostate cancer and predict clinical relapse. The American journal of pathology 2012; 180(6):2240-8.
6. Yu Y P, Ding Y, Chen R, Liao S G, Ren B G, Michalopoulos A, et al. Whole-Genome Methylation Sequencing Reveals Distinct Impact of Differential Methylations on Gene Transcription in Prostate Cancer. The American journal of pathology 2013.
7. Luo J H, Ding Y, Chen R, Michalopoulos G, Nelson J, Tseng G, et al. Genome-wide methylation analysis of prostate tissues reveals global methylation patterns of prostate cancer. The American journal of pathology 2013; 182(6):2028-36.
8. Zuo Z H, Yu Y P, Ren B G, Liu S, Nelson J, Wang Z, et al. Oncogenic Activity of Solute Carrier Family 45 Member 2 and Alpha-Methylacyl-Coenzyme A Racemase Gene Fusion Is Mediated by Mitogen-Activated Protein Kinase. Hepatol Commun 2022; 6(1):209-22 doi 10.1002/hep4.1724.
9. Chen Z H, Yu Y P, Tao J, Liu S, Tseng G, Nalesnik M, et al. MAN2A1-FER Fusion Gene Is Expressed by Human Liver and Other Tumor Types and Has Oncogenic Activity in Mice. Gastroenterology 2017; 153(4):1120-32 doi 10.1053/j.gastro.2016.12.036.
10. Lin F, Yu Y P, Woods J, Cieply K, Gooding B, Finkelstein P, et al. Myopodin, a synaptopodin homologue, is frequently deleted in invasive prostate cancers. American Journal of Pathology 2001; 159(5):1603-12.
11. Yu Y P, Landsittel D, Jing L, Nelson J, Ren B, Liu L, et al. Gene expression alterations in prostate cancer predicting tumor aggression and preceding development of malignancy. J Clin Oncol 2004; 22(14):2790-9.
12. Luo J H, Ren B, Keryanov S, Tseng G C, Rao U N, Monga S P, et al. Transcriptomic and genomic analysis of human hepatocellular carcinomas and hepatoblastomas. Hepatology (Baltimore, Md 2006; 44(4):1012-24.
13. He D M, Ren B G, Liu S, Tan L Z, Cieply K, Tseng G, et al. Oncogenic activity of amplified miniature chromosome maintenance 8 in human malignancies. Oncogene 2017; 36(25):3629-39 doi 10.1038/onc.2017.123.
14. Yu Y P, Liu P, Nelson J, Hamilton R L, Bhargava R, Michalopoulos G, et al. Identification of recurrent fusion genes across multiple cancer types. Sci Rep 2019; 9(1):1074 doi 10.1038/s41598-019-38550-6.
15. Cortes C, Vapnik V. Support-vector networks. Machine Learning 1995; 20:273-97 doi https://doi.org/10.1007/BF00994018.
16. Amit Y, Geman D. Shape quantization and recognition with randomized trees. Neural computation 1997; 9.7(1545-1588).
17. Bauer E, Kohavi R. An empirical comparison of voting classification algorithms: Bagging, boosting, and variants. Machine Learning 1999; 36:105-39.
18. McLachlan G J. Discriminant analysis and statistical pattern recognition. Applied Probability & Statistics 2004:1-526.
19. Tolles J, Meurer W J. Logistic Regression: Relating Patient Characteristics to Outcomes. JAMA 2016; 316(5):533-4 doi 10.1001/jama.2016.7653.
20. Rosenkrantz A B, Oto A, Turkbey B, Westphalen A C: Prostate Imaging Reporting and Data System (PI-RADS), version 2: a critical look. AJR Am J Roentgenol 2016, 206:1179e1183
21. Den R B, Santiago-Jimenez M, Alter J, Schliekelman M, Wagner J R, Renzulli Ii J F, Lee D I, Brito C G, Monahan K, Gburek B, Kella N, Vallabhan G, Abdollah F, Trabulsi E J, Lallas C D, Gomella L G, Woodlief T L, Haddad Z, Lam L L, Deheshi S, Wang Q, Choeurng V, du Plessis M, Jordan J, Parks B, Shin H, Buerki C, Yousefi K, Davicioni E, Patel V R, Shah N L: Decipher correlation patterns post prostatectomy: initial experience from 2342 prospective patients. Prostate Cancer Prostatic Dis 2016, 19:374e379
22. Makela T P, Parvin J D, Kim J, Huber L J, Sharp P A, Weinberg R A. A kinase-deficient transcription factor TFIIH is functional in basal and activated transcription. Proceedings of the National Academy of Sciences of the United States of America 1995; 92(11):5174-8.
23. Fisher R P, Morgan D O. A novel cyclin associates with MO15/CDK7 to form the CDK-activating kinase. Cell 1994; 78(4):713-24.
24. Shiekhattar R, Mermelstein F, Fisher R P, Drapkin R, Dynlacht B, Wessling H C, et al. Cdk-activating kinase complex is a component of human transcription factor TFIIH. Nature 1995; 374(6519):283-7.
25. Andersen G, Busso D, Poterszman A, Hwang J R, Wurtz J M, Ripp R, et al. The structure of cyclin H: common mode of kinase activation and specific features. The EMBO journal 1997; 16(5):958-67 doi 10.1093/emboj/16.5.958.
26. Chen Z H, Yu Y P, Zuo Z H, Nelson J B, Michalopoulos G K, Monga S, et al. Targeting genomic rearrangements in tumor cells through Cas9-mediated insertion of a suicide gene. Nature biotechnology 2017; 35(6):543-50 doi 10.1038/nbt.3843.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. A method of determining whether a subject is at risk of prostate cancer recurrence, the method comprising:
(a) obtaining a sample from a subject;
(b) detecting one or more fusion genes in the sample;
(c) generating a probability score by a machine learning model, based on an analysis of the one or more fusion genes with respect to fusion genes associated with a reference population; and
(d) determining, based on the probability score, the risk of prostate cancer recurrence in the subject.

2. The method of claim 1, wherein the sample is a blood sample, a serum sample, or a tumor sample.

3. The method of claim 2, wherein the sample is processed for RNA isolation.

4. The method of claim 3, wherein the detection of at least one fusion gene, is determined by reverse transcription polymerase chain reaction (RT-PCR).

5. The method of claim 1, wherein one or more fusion genes is selected from the group consisting of MAN2A1-FER, TRMT11-GRIK2, MTOR-TP53BP1, CCNH-05orf30, KDM4B-AC011523.2, SLC45A2-AMACR, TMEM135-CCDC67, LRRC59-FLJ60017, CLTC-ETV1, PCMTD1-SNTG1, ACPP-SEC13, DOCK7-OLR1, ZMPSTE24-ZMYM4, Pten-NOLC1, and combinations thereof.

6. The method of claim 1, wherein the machine learning model comprises one or more machine learning algorithms selected from the group consisting of support vector machine (SVM), random forest (RF), linear discriminant analysis (LDA), logistic regression, or any combination thereof.

7. The method of claim 1, wherein a Gleason score or serum PSA level, or combination of both, of the subject are incorporated into the machine learning model.

8. The method of claim 7, wherein fusion gene status, Gleason score, PSA level are incorporated into the machine learning model based on leave-one-out cross-validation (LOOCV) analysis of a plurality of training data.

9. The method of claim 7, wherein the Gleason score incorporated into the machine learning model is assigned a Gleason cutoff value of 8.

10. The method of claim 7, wherein the PSA level incorporated into the machine learning model is assigned a PSA level cutoff value of 9.77 ng/mL.

11. The method of claim 1, where the subject has received radical prostatectomy or radiation therapy.

12. The method of claim 10, wherein the subject has not received radiation or hormone therapy prior to radical prostatectomy.

13. The method of claim 1, wherein if the probability score is equal to or less than 0.5 the prostate cancer is predicted as non-recurrent.

14. The method of claim 1, wherein if the probability score is more than 0.5 the prostate cancer is predicted as recurrent.

15. The method of claim 1, wherein the machine learning model comprises one or more neural networks.

16. The method of claim 1, further comprising:
accessing, by the machine learning model, prostate cancer imaging data of the subject, wherein generating the prediction score is further based on an additional analysis of the prostate cancer imaging data by the machine learning model.

17. The method of claim 1, further comprising:
accessing, by the machine learning model, biomedical imaging data of the subject, wherein the biomedical imaging data comprises one or more of MRI data, X-ray data, ultrasound data, or any combination thereof, and wherein generating the prediction score is further based on an additional analysis of the biomedical imaging data by the machine learning model.

18. The method of claim 1, further comprising:
accessing, by the machine learning model, an output from a prostate genome deciper classifier, wherein generating the prediction score is further based on the output from the prostate genome deciper.


SEQUENCE LISTING (45 sequences)

SEQ ID NO: 1 - DNA, 21 bp, synthetic construct
  misc_feature 1..21: note = Description of sequence: Primer. Forward Primer, Name: MAN2A1-FER
  source 1..21: mol_type = other DNA
agcgcagtttgggatacagca

SEQ ID NO: 2 - DNA, 27 bp, synthetic construct
  misc_feature 1..27: note = Description of sequence: Primer. Reverse Primer, Name: MAN2A1-FER
  source 1..27: mol_type = other DNA
ctttaatgtgcccttatatacttcacc

SEQ ID NO: 3 - DNA, 25 bp, synthetic construct
  misc_feature 1..25: note = Description of sequence: Probe Name: MAN2A1-FER
  source 1..25: mol_type = other DNA
  modified_base 1: mod_base = OTHER; note = bound to dye, specific dye: 5'-carboxyfluorescein (6-FAM)
  modified_base 9: mod_base = OTHER; note = bound to dye, specific dye: ZEN
  modified_base 25: mod_base = OTHER; note = bound to dye, specific dye: Iowa black fluorescein quencher
tcagaaacagcctatgagggaaatt

SEQ ID NO: 4 - DNA, 21 bp, synthetic construct
  misc_feature 1..21: note = Description of sequence: Primer. Forward Primer, Name: SLC45A2-AMACR
  source 1..21: mol_type = other DNA
ttgatgtctgctcccatcagg

SEQ ID NO: 5 - DNA, 21 bp, synthetic construct
  misc_feature 1..21: note = Description of sequence: Primer. Reverse Primer, Name: SLC45A2-AMACR
  source 1..21: mol_type = other DNA
cagctggagtttctccatgac

SEQ ID NO: 6 - DNA, 22 bp, synthetic construct
  misc_feature 1..22: note = Description of sequence: Probe Name: SLC45A2-AMACR
  source 1..22: mol_type = other DNA
  modified_base 1: mod_base = OTHER; note = bound to dye, specific dye: 5'-carboxyfluorescein (6-FAM)
  modified_base 9: mod_base = OTHER; note = bound to dye, specific dye: ZEN
  modified_base 22: mod_base = OTHER; note = bound to dye, specific dye: Iowa black fluorescein quencher
aagagggcatggtagtggaggc

SEQ ID NO: 7 - DNA, 29 bp, synthetic construct
  misc_feature 1..29: note = Description of sequence: Primer. Forward Primer, Name: CCNH-05orf30
  source 1..29: mol_type = other DNA
aaagttatttatcagagagtctgatgctg

SEQ ID NO: 8 - DNA, 30 bp, synthetic construct
  misc_feature 1..30: note = Description of sequence: Primer. Reverse Primer, Name: CCNH-05orf30
  source 1..30: mol_type = other DNA
ctgttctactccaggtattttcattatatc

SEQ ID NO: 9 - DNA, 27 bp, synthetic construct
  misc_feature 1..27: note = Description of sequence: Probe Name: CCNH-05orf30
  source 1..27: mol_type = other DNA
  modified_base 1: mod_base = OTHER; note = bound to dye, specific dye: 5'-carboxyfluorescein (6-FAM)
  modified_base 9: mod_base = OTHER; note = bound to dye, specific dye: ZEN
  modified_base 27: mod_base = OTHER; note = bound to dye, specific dye: Iowa black fluorescein quencher
acaggcaagttctgttctctttcagca

SEQ ID NO: 10 - DNA, 21 bp, synthetic construct
  misc_feature 1..21: note = Description of sequence: Primer. Forward Primer, Name: MTOR-TP53BP1
  source 1..21: mol_type = other DNA
tgatagaccagtcccgggatg

SEQ ID NO: 11 - DNA, 22 bp, synthetic construct
  misc_feature 1..22: note = Description of sequence: Primer. Reverse Primer, Name: MTOR-TP53BP1
  source 1..22: mol_type = other DNA
ccactgacattcccagaacaag

SEQ ID NO: 12 - DNA, 27 bp, synthetic construct
  misc_feature 1..27: note = Description of sequence: Probe Name: MTOR-TP53BP1
  source 1..27: mol_type = other DNA
  modified_base 1: mod_base = OTHER; note = bound to dye, specific dye: 5'-carboxyfluorescein (6-FAM)
  modified_base 9: mod_base = OTHER; note = bound to dye, specific dye: ZEN
  modified_base 27: mod_base = OTHER; note = bound to dye, specific dye: Iowa black fluorescein quencher
tgtcagcctgtcagaatccaagtcaag

SEQ ID NO: 13 - DNA, 21 bp, synthetic construct
  misc_feature 1..21: note = Description of sequence: Primer. Forward Primer, Name: TRMT11-GRIK2
  source 1..21: mol_type = other DNA
cccttaacaggtatctgctcc

SEQ ID NO: 14 - DNA, 21 bp, synthetic construct
  misc_feature 1..21: note = Description of sequence: Primer. Reverse Primer, Name: TRMT11-GRIK2
  source 1..21: mol_type = other DNA
cccattgggccagattccaca

SEQ ID NO: 15 - DNA, 22 bp, synthetic construct
  misc_feature 1..22: note = Description of sequence: Probe Name: TRMT11-GRIK2
  source 1..22: mol_type = other DNA
  modified_base 1: mod_base = OTHER; note = bound to dye, specific dye: 5'-carboxyfluorescein (6-FAM)
  modified_base 9: mod_base = OTHER; note = bound to dye, specific dye: ZEN
  modified_base 22: mod_base = OTHER; note = bound to dye, specific dye: Iowa black fluorescein quencher
cggaactccagatgctcctgcg

SEQ ID NO: 16 - DNA, 21 bp, synthetic construct
  misc_feature 1..21: note = Description of sequence: Primer. Forward Primer, Name: LRRC59-FL.160017
  source 1..21: mol_type = other DNA
gtgactgcttggatgagaagc

SEQ ID NO: 17 - DNA, 21 bp, synthetic construct
  misc_feature 1..21: note = Description of sequence: Primer. Reverse Primer, Name: LRRC59-FL.160017
  source 1..21: mol_type = other DNA
gttgatgagcagccattgagc

SEQ ID NO: 18 - DNA, 26 bp, synthetic construct
  misc_feature 1..26: note = Description of sequence: Probe Name: LRRC59-FL.160017
  source 1..26: mol_type = other DNA
  modified_base 1: mod_base = OTHER; note = bound to dye, specific dye: 5'-carboxyfluorescein (6-FAM)
  modified_base 9: mod_base = OTHER; note = bound to dye, specific dye: ZEN
  modified_base 26: mod_base = OTHER; note = bound to dye, specific dye: Iowa black fluorescein quencher
cagtgtgcaaacaaggtgactggaag

SEQ ID NO: 19 - DNA, 23 bp, synthetic construct
  misc_feature 1..23: note = Description of sequence: Primer. Forward Primer, Name: TMEM135-CCDC67
  source 1..23: mol_type = other DNA
gagaccatcttactggaagttcc

SEQ ID NO: 20 - DNA, 21 bp, synthetic construct
  misc_feature 1..21: note = Description of sequence: Primer. Reverse Primer, Name: TMEM135-CCDC67
  source 1..21: mol_type = other DNA
tggtactcttccacctgttgg

SEQ ID NO: 21 - DNA, 28 bp, synthetic construct
  misc_feature 1..28: note = Description of sequence: Probe Name: TMEM135-CCDC67
  source 1..28: mol_type = other DNA
  modified_base 1: mod_base = OTHER; note = bound to dye, specific dye: 5'-carboxyfluorescein (6-FAM)
  modified_base 9: mod_base = OTHER; note = bound to dye, specific dye: ZEN
  modified_base 28: mod_base = OTHER; note = bound to dye, specific dye: Iowa black fluorescein quencher
tttgcccttggtgagtcttaaaaggaac

SEQ ID NO: 22 - DNA, 20 bp, synthetic construct
  misc_feature 1..20: note = Description of sequence: Primer. Forward Primer, Name: KDM4B-AC011523.2
  source 1..20: mol_type = other DNA
cacaccgaggacatggacct

SEQ ID NO: 23 - DNA, 21 bp, synthetic construct
  misc_feature 1..21: note = Description of sequence: Primer. Reverse Primer, Name: KDM4B-AC011523.2
  source 1..21: mol_type = other DNA
ctcagatccaggcttgcttac

SEQ ID NO: 24 - DNA, 26 bp, synthetic construct
  misc_feature 1..26: note = Description of sequence: Probe Name: KDM4B-AC011523.2
  source 1..26: mol_type = other DNA
  modified_base 1: mod_base = OTHER; note = bound to dye, specific dye: 5'-carboxyfluorescein (6-FAM)
  modified_base 9: mod_base = OTHER; note = bound to dye, specific dye: ZEN
  modified_base 26: mod_base = OTHER; note = bound to dye, specific dye: Iowa black fluorescein quencher
acagcatcaactacctgcactttggg

SEQ ID NO: 25 - DNA, 22 bp, synthetic construct
  misc_feature 1..22: note = Description of sequence: Primer. Forward Primer, Name: CLTC-ETV1
  source 1..22: mol_type = other DNA
ccttcctcctacatggaagttg

SEQ ID NO: 26 - DNA, 21 bp, synthetic construct
  misc_feature 1..21: note = Description of sequence: Primer. Reverse Primer, Name: CLTC-ETV1
  source 1..21: mol_type = other DNA
cttgattttcagtggcaggcc

SEQ ID NO: 27 - DNA, 23 bp, synthetic construct
  misc_feature 1..23: note = Description of sequence: Probe Name: CLTC-ETV1
  source 1..23: mol_type = other DNA
  modified_base 1: mod_base = OTHER; note = bound to dye, specific dye: 5'-carboxyfluorescein (6-FAM)
  modified_base 9: mod_base = OTHER; note = bound to dye, specific dye: ZEN
  modified_base 23: mod_base = OTHER; note = bound to dye, specific dye: Iowa black fluorescein quencher
ctgccaatactagtgtggctttt

SEQ ID NO: 28 - DNA, 23 bp, synthetic construct
  misc_feature 1..23: note = Description of sequence: Primer. Forward Primer, Name: PCMTD1-SNTG1
  source 1..23: mol_type = other DNA
ctggagagcttcatcaaaaatag

SEQ ID NO: 29 - DNA, 22 bp, synthetic construct
  misc_feature 1..22: note = Description of sequence: Primer. Reverse Primer, Name: PCMTD1-SNTG1
  source 1..22: mol_type = other DNA
cacttctcgggcaatctcaaca

SEQ ID NO: 30 - DNA, 30 bp, synthetic construct
  misc_feature 1..30: note = Description of sequence: Probe Name: PCMTD1-SNTG1
  source 1..30: mol_type = other DNA
  modified_base 1: mod_base = OTHER; note = bound to dye, specific dye: 5'-carboxyfluorescein (6-FAM)
  modified_base 9: mod_base = OTHER; note = bound to dye, specific dye: ZEN
  modified_base 30: mod_base = OTHER; note = bound to dye, specific dye: Iowa black fluorescein quencher
agctttgataaactgctctccagaatgttg

SEQ ID NO: 31 - DNA, 21 bp, synthetic construct
  misc_feature 1..21: note = Description of sequence: Primer. Forward Primer, Name: ACPP-SEC13
  source 1..21: mol_type = other DNA
cctcatggccacaaggatttg

SEQ ID NO: 32 - DNA, 21 bp, synthetic construct
  misc_feature 1..21: note = Description of sequence: Primer. Reverse Primer, Name: ACPP-SEC13
  source 1..21: mol_type = other DNA
tgaggcttccaggtacaacag

SEQ ID NO: 33 - DNA, 22 bp, synthetic construct
  misc_feature 1..22: note = Description of sequence: Probe Name: ACPP-SEC13
  source 1..22: mol_type = other DNA
  modified_base 1: mod_base = OTHER; note = bound to dye, specific dye: 5'-carboxyfluorescein (6-FAM)
  modified_base 9: mod_base = OTHER; note = bound to dye, specific dye: ZEN
  modified_base 22: mod_base = OTHER; note = bound to dye, specific dye: Iowa black fluorescein quencher
ccagattggctgcaatgccgtc

SEQ ID NO: 34 - DNA, 25 bp, synthetic construct
  misc_feature 1..25: note = Description of sequence: Primer. Forward Primer, Name: DOCK7-0LR1
  source 1..25: mol_type = other DNA
taaaacaagggtcaatgtcactcat

SEQ ID NO: 35 - DNA, 23 bp, synthetic construct
  misc_feature 1..23: note = Description of sequence: Primer. Reverse Primer, Name: DOCK7-0LR1
  source 1..23: mol_type = other DNA
cagtctggatctttaggtcatca

SEQ ID NO: 36 - DNA, 21 bp, synthetic construct
  misc_feature 1..21: note = Description of sequence: Probe Name: DOCK7-0LR1
  source 1..21: mol_type = other DNA
  modified_base 1: mod_base = OTHER; note = bound to dye, specific dye: 5'-carboxyfluorescein (6-FAM)
  modified_base 9: mod_base = OTHER; note = bound to dye, specific dye: ZEN
  modified_base 21: mod_base = OTHER; note = bound to dye, specific dye: Iowa black fluorescein quencher
agacacagcaggatgccaatg

SEQ ID NO: 37 - DNA, 23 bp, synthetic construct
  misc_feature 1..23: note = Description of sequence: Primer. Forward Primer, Name: ZMPSTE24-ZMYM4
  source 1..23: mol_type = other DNA
gaggaagaagggaacagtgaaga

SEQ ID NO: 38 - DNA, 29 bp, synthetic construct
  misc_feature 1..29: note = Description of sequence: Primer. Reverse Primer, Name: ZMPSTE24-ZMYM4
  source 1..29: mol_type = other DNA
ctggaatagggctcagtaaaaatgttatc

SEQ ID NO: 39 - DNA, 21 bp, synthetic construct
  misc_feature 1..21: note = Description of sequence: Probe Name: ZMPSTE24-ZMYM4
  source 1..21: mol_type = other DNA
  modified_base 1: mod_base = OTHER; note = bound to dye, specific dye: 5'-carboxyfluorescein (6-FAM)
  modified_base 9: mod_base = OTHER; note = bound to dye, specific dye: ZEN
  modified_base 21: mod_base = OTHER; note = bound to dye, specific dye: Iowa black fluorescein quencher
agacacagcaggatgccaatg

SEQ ID NO: 40 - DNA, 23 bp, synthetic construct
  misc_feature 1..23: note = Description of sequence: Primer. Forward Primer, Name: PTEN-NOLC1
  source 1..23: mol_type = other DNA
aagccaaccgatacttttctcca

SEQ ID NO: 41 - DNA, 23 bp, synthetic construct
  misc_feature 1..23: note = Description of sequence: Primer. Reverse Primer, Name: PTEN-NOLC1
  source 1..23: mol_type = other DNA
atagatgtctaagagggaagagg

SEQ ID NO: 42 - DNA, 21 bp, synthetic construct
  misc_feature 1..21: note = Description of sequence: Probe Name: PTEN-NOLC1
  source 1..21: mol_type = other DNA
  modified_base 1: mod_base = OTHER; note = bound to dye, specific dye: 5'-carboxyfluorescein (6-FAM)
  modified_base 9: mod_base = OTHER; note = bound to dye, specific dye: ZEN
  modified_base 21: mod_base = OTHER; note = bound to dye, specific dye: Iowa black fluorescein quencher
agacacagcaggatgccaatg

SEQ ID NO: 43 - DNA, 21 bp, synthetic construct
  misc_feature 1..21: note = Description of sequence: Primer. Forward Primer, Name: ACTS
  source 1..21: mol_type = other DNA
accccacttctctctaaggag

SEQ ID NO: 44 - DNA, 21 bp, synthetic construct
  misc_feature 1..21: note = Description of sequence: Primer. Reverse Primer, Name: ACTS
  source 1..21: mol_type = other DNA
gcaatgctatcacctcccctg

SEQ ID NO: 45 - DNA, 23 bp, synthetic construct
  misc_feature 1..23: note = Description of sequence: Probe Name: ACTS
  source 1..23: mol_type = other DNA
  modified_base 1: mod_base = OTHER; note = bound to dye, specific dye: 5'-carboxyfluorescein (6-FAM)
  modified_base 9: mod_base = OTHER; note = bound to dye, specific dye: ZEN
  modified_base 23: mod_base = OTHER; note = bound to dye, specific dye: Iowa black fluorescein quencher
ccagtcctctcccaagtccacac